SEQUENCE LISTING (2 sequences)

SEQ ID NO: 1 - protein, 37 aa, Artificial
  Synthetically generated peptide
YTSLIHSLIEESQNQQEKNEQELLELDKWASLWNWFC

SEQ ID NO: 2 - protein, 36 aa, Artificial
  Synthetically generated peptide
YTSLIHSLIEESQNQQEKNEQELLELDKWASLWNWF

(19) United States
(12) Patent Application Publication
(10) Pub. No.: US 2007/0122408 A1
(43) Pub. Date: May 31, 2007

(54) Fc Labeling for Immunostaining and Immunotargeting

(75) Inventor: Carlos F. Barbas III, La Jolla, CA (US)

Correspondence Address: CATALYST LAW GROUP, APC, 9710 SCRANTON ROAD, SUITE S-170, SAN DIEGO, CA, 92121, US

(73) Assignee: The Scripps Research Institute, La Jolla, CA (US)

(21) Appl. No.: 11/551,665

(22) Filed: Oct. 20, 2006

Related U.S. Application Data
(60) Provisional application No. 60/728,821, filed on Oct. 20, 2005.

(51) Int. Cl. A61K 39/395; C12P 21/06; C07K 16/28; C07K 16/46

(52) U.S. Cl. 424/144.1; 424/178.1; 435/68.1; 530/391.1; 530/388.22

(57) ABSTRACT

The present invention discloses methods of labeling Fc portions of antibodies, or fusion proteins incorporating Fc portions of antibodies, so that they can be used in immunostaining or immunolabeling procedures. A wide variety of labels can be used. A linker can be used between the label and the protein to be labeled, allowing for flexibility in labeling. A large variety of coupling reactions can be used to generate the labeled protein molecule, The protein molecule to be labeled can be part of a larger fusion protein. The labeled protein molecules can be used in immunostaining and immunolabeling procedures but also in in vivo applications for therapy and diagnostic imaging.

### CROSS-REFERENCES

This application claims priority from U. S. Provisional Application Ser. No. 60/728,821, by Carlos F. Barbas III, entitled “Fc Labeling for Immunostaining and Immunotargeting,” and filed Oct. 20, 2005, which is incorporated herein in its entirety by this reference.

### FIELD OF THE INVENTION

This invention is directed to methods of labeling the Fc portion of antibody molecules and related molecules including Fc regions for immunostaining and immunotargeting.

Antibodies are biological macromolecules with highly defined specificity. This specificity arises from the unique way the antibodies are generated. The use of antibody molecules in immunoassay, immunostaining, or immunotargeting encompasses a broad variety of applications, including in in vitro immunohistochemistry or immunocytochemistry and in in vivo labeling and detection.

Naturally-occurring immunoglobulins are tetramers with the general structure L₂H₂, with L being a so-called “light chain,” typically with a molecular weight of about 25,000 and H being a so-called “heavy chain,” typically with a molecular weight of 50,000. In naturally-occurring immunoglobulins, the two light chains and the two heavy chains are identical; these chains are held together by interchain disulfide bonds. Intrachain disulfide bonds also contribute to the stability of the antibody molecule.

Immunoglobulins are divided into classes depending on the type of heavy chain found therein. The possible heavy chain molecules are designated γ, μ, α, ε, and δ, which give rise to immunoglobulins of class IgG, IgM, IgA, IgE, and IgD, respectively. Of these classes, the most common and the most frequently utilized is IgG. The discussion below therefore focuses on IgG immunoglobulins, with the understanding that it is also applicable to immunoglobulins of other classes unless excluded.

In immunoglobulins, such as IgG, there are regions or domains that provide specific functions. The presence of these domains is a consequence of the structure of the molecule. Both heavy chains and light chains include variable (V) regions and constant (C) regions. The antigen-binding site includes only a portion of the variable regions of both H and L chains, which include the actual amino acids responsible for the specific binding of the corresponding antigen by the antibody; these amino acids are referred to as the hypervariable region or the complementarity-determining regions (CDRs). The V regions include the amino-terminal portions of both H and L chains. The carboxyl-terminal portion of the H chains forms a region known as Fc. The Fc region plays no direct role in antigen binding, but is responsible for a number of effector functions, such as complement fixation and the generation of antibody-dependent cellular cytotoxicity (ADCC), as well as the half-life in circulation.

Therefore, there is a particular need for methods that can be used for modifying antibody molecules in the Fc regions to produce reagents that can be used for immunostaining or immunotargeting without interfering with the antigen-binding specificity of the antibody molecules. These reagents should include reagents that target cellular or extracellular proteins, such as integrins, as well as other biologically significant molecules, in such a way that the reagents can be used for therapeutic as well as diagnostic purposes. Preferably, such methods that can be used to modify antibody molecules do so in a manner that preserves the activity of the Fc region, such as effector functions and circulatory half-life.

### SUMMARY OF THE INVENTION

One aspect of the invention is a method for labeling a protein molecule that includes therein the Fc portion of an antibody molecule comprising the steps of:
(1) providing a protein molecule that includes therein the Fc portion of an antibody molecule, the molecule having an amino-terminal serine residue;
(2) oxidizing the amino-terminal serine residue to an aldehyde group; and
(3) reacting the protein molecule with a targeting molecule including therein a moiety reactive with an aldehyde to produce a labeled protein molecule such that the targeting molecule solely directs the targeting of the labeled protein molecule to a target that is a soluble molecule or a cell-surface molecule.

Another aspect of the invention is a method for labeling a protein molecule that includes therein the Fc portion of an antibody molecule comprising the steps of:
(1) providing a protein molecule that includes therein the Fc portion of an antibody molecule, the molecule having at least one amino acid including therein a side chain with aldehyde or keto functionality; and
(2) reacting the aldehyde or keto functionality of the protein molecule with a targeting molecule including therein a group reactive with an aldehyde or keto functionality to produce a labeled protein molecule such that the targeting molecule solely directs the targeting of the labeled protein molecule to a target that is a soluble molecule or a cell-surface molecule.

Yet another aspect of the invention is a method for labeling a protein molecule that includes therein the Fc portion of an antibody molecule comprising the steps of:
(1) providing a protein molecule that includes therein the Fc portion of an antibody molecule, the protein molecule having a reactive amino acid residue selected from the group consisting of an azide-substituted amino acid residue and an alkyne-substituted amino acid residue;
(2) providing a targeting molecule, the targeting molecule having a reactive residue selected from the group consisting of an azide and an alkyne such that the protein molecule and the targeting molecule, taken together, have an azide modification and an alkyne modification; and
(3) reacting the protein molecule with the targeting molecule by azide-alkyne [3+2] cycloaddition to produce a labeled protein molecule such that the targeting molecule solely directs the targeting of the labeled protein molecule to a target that is a soluble molecule or a cell-surface molecule.

Yet another aspect of the invention is a method for labeling a protein molecule that includes therein the Fc portion of an antibody molecule comprising the steps of:
(1) providing a protein molecule that includes therein the Fc portion of an antibody molecule, the protein molecule having a reactive aldehyde residue;
(2) reacting the aldehyde residue with a bifunctional hydroxylamine linker having two H₂N—O— moieties, the aldehyde residue forming a C═N bond with one of the moieties; and
(3) reacting the other H₂N—O— moiety of the bifunctional hydroxylamine linker with a targeting molecule having a diketone moiety to produce a labeled protein molecule such that the targeting molecule solely directs the targeting of the labeled protein molecule to a target that is a soluble molecule or a cell-surface molecule.

Still another aspect of the invention is a method for labeling a protein molecule that includes therein the Fc portion of an antibody molecule comprising the steps of:
(1) providing a protein molecule that includes therein the Fc portion of an antibody molecule, the molecule having at least one amino acid including therein a side chain with azido functionality; and
(2) in a Staudinger ligation reaction, reacting the azido functionality of the protein molecule with a targeting molecule that is covalently linked to an ortho-disubstituted aromatic moiety, one substituent being carbomethoxy and the other substitutent being diphenylphosphino, to produce a labeled protein molecule, such that the labeled protein molecule has one substituent of the aromatic moiety being diphenylphosphinyl and the other substituent being a carboxamide moiety, with the nitrogen of the carboxamide moiety being linked to the protein molecule such that the targeting molecule solely directs the targeting of the labeled protein molecule to a target that is a soluble molecule or a cell-surface molecule.

Yet another aspect of the invention is a method for labeling a protein molecule that includes therein the Fc portion of an antibody molecule comprising the steps of:
(1) providing a protein molecule that includes therein the Fc portion of an antibody molecule, the molecule having an amino acid selected from the group consisting of p-acetylphenylalanine and m-acetylphenylalanine; and

(2) reacting the amino acid selected from the group consisting of p-acetyiphenylalanine and m-acetylphenylalanine of the protein molecule with a targeting molecule containing a reactive moiety selected from the group consisting of a hydrazide, an alkoxyamine, and a semicarbazide to produce a labeled protein molecule such that the targeting molecule solely directs the targeting of the labeled protein molecule to a target that is a soluble molecule or a cell-surface molecule.

Still another aspect of the invention is a method for labeling a protein molecule that includes therein the Fc portion of an antibody molecule comprising the steps of:
(1) providing a protein molecule that includes therein the Fc portion of an antibody molecule, the protein molecule having a reactive amino acid residue reactive with an electrophile;
(2) providing a targeting molecule that includes an electrophile reactive with the amino acid residue; and
(3) reacting the targeting molecule with the protein molecule by reacting the reactive amino acid residue with the electrophile to produce the labeled protein molecule such that the targeting molecule solely directs the targeting of the labeled protein molecule to a target that is a soluble molecule or a cell-surface molecule.

Yet another aspect of the invention is a method for labeling a protein molecule that includes therein the Fc portion of an antibody molecule comprising the steps of:
(1) providing a protein molecule that includes therein the Fc portion of an antibody molecule, the protein molecule having a reactive amino acid residue including therein an electrophilic group reactive with a nucleophile;
(2) providing a targeting molecule that includes a nucleophile reactive with the amino acid residue; and
(3) reacting the targeting molecule with the protein molecule by reacting the reactive amino acid residue with the nucleophile to produce the labeled protein molecule such that the targeting molecule solely directs the targeting of the labeled protein molecule to a target that is a soluble molecule or a cell-surface molecule.

Still another aspect of the invention is a method for labeling a protein molecule that includes therein the Fc portion of an antibody molecule comprising the steps of:
(1) providing a protein molecule that includes therein the Fc portion of an antibody molecule, the protein molecule having a mutated haloalkane dehalogenase domain therein, the mutated haloalkane dehalogenase domain having therein an aspartate residue, the side chain of the aspartate residue being capable of esterification; and
(2) reacting the protein molecule with a targeting molecule having a reactive haloalkane moiety to form a stable ester to produce a labeled protein molecule such that the targeting molecule solely directs the targeting of the labeled protein molecule to a target that is a soluble molecule or a cell-surface molecule.

In still another general labeling method according to the present invention, the method comprises the steps of:
(1) providing a protein molecule that includes therein the Fc portion of an antibody molecule, the protein molecule having a first reactive amino acid at its amino-terminus and a second reactive amino acid at its carboxyl-terminus;
(2) reacting a first molecule selected from the group consisting of a targeting molecule and a component of a fusion protein with the first reactive amino acid to link the first molecule to the protein molecule; and
(3) reacting a second molecule selected from the group consisting of a targeting molecule and a component of a fusion protein with the second reactive amino acid to link the second molecule to the protein molecule;
   with the proviso that the first reactive amino acid does not react with the second reactive amino acid such that the targeting molecule solely directs the targeting of the labeled protein molecule to a target that is a soluble molecule or a cell-surface molecule

The protein molecule to be labeled can include various segments of the Fc region and can be part of a larger fusion protein.

In one alternative, the targeting molecule comprises: (a) a targeting module; (b) a linker covalently linked to the targeting module; and (c) a reactive module covalently linked to the linker, the reactive module including therein a hydroxylamine moiety or derivative thereof or another reactive moiety as appropriate to react with the protein.

In another alternative, the targeting molecule comprises: (a) a targeting module; and (b) a reactive module covalently linked to the targeting module, the reactive module including therein a hydroxylamine moiety or derivative thereof or another reactive moiety as appropriate to react with the protein.

In one preferred alternative, the targeting module specifically targets an integrin. The targeting module can be a peptidomimetic such as a RGD peptidomimetic. The targeting module can alternatively target another peptide, another protein, or another biomolecule. For example, the targeting module can be modified T-20 peptide having the amino acid sequence N-Acetyl-YTSLIHSLIEESQNQQEKNE QELLELDKWASLWNWFC (SEQ ID NO: 1), which can act as an inhibitor of HIV-1 infection.

In another alternative, the targeting module comprises a label. Various labels can be used, including secondary labeling.

If used, typically the linker has the general structure X—Z wherein X is a linear or branched connecting chain of atoms comprising any of C, H, N, O, P, S, Si, F, CI, Br, and I, or a salt thereof, and comprising a repeating ether unit of between 2-100 units; and Z is a hydroxylamine moiety or other reactive moiety as appropriate to react with the protein.

The labeled protein can be glycosylated and can substantially maintain its naturally-occurring pattern of glycosylation.

Another aspect of the invention is a mutated protein including the Fc portion of an antibody molecule incorporating an altered amino acid at its amino-terminus to provide reactivity with a targeting molecule as described above, or incorporating a non-naturally-occurring amino acid.

More generally, yet another aspect of the invention is a mutated protein including the Fc portion of an antibody molecule and incorporating therein a non-naturally-occurring amino acid, the non-naturally-occurring amino acid being selected from the group consisting of:
(1) an azide-substituted amino acid;
(2) an alkyne-substituted amino acid;
(3) p-acetylphenylalanine;
(4) m-acetylphenylalanine;
(5) β-oxo-α-aminobutyric acid; and
(6) (2-ketobutyl)-tyrosine;
   wherein the non-naturally-occurring amino acid is located such that the mutated protein can be covalently linked to a targeting molecule such that the targeting molecule solely directs the targeting of the mutated protein molecule to a target that is a soluble molecule or a cell-surface molecule.

Still more generally, another aspect of the invention is a mutated protein comprising a protein selected from the group consisting of:
(1) a mutated protein including the Fc portion of an antibody molecule therein and incorporating an altered amino acid at the amino-terminus of the sequence of the protein and differing from the naturally-occurring protein by no more than two conservative amino acid substitutions exclusive of the alteration of the amino acid at the amino-terminus; and
(2) a mutated protein including the Fc portion of an antibody molecule therein and incorporating therein a non-naturally-occurring amino acid, the non-naturally-occurring amino acid being selected from the group consisting of:
   (a) an azide-substituted amino acid;
   (b) an alkyne-substituted amino acid;
   (c) p-acetylphenylalanine;
   (d) m-acetylphenylalanine;
   (e) β-oxo-α-aminobutyric acid; and
   (f) (2-ketobutyl)-tyrosine;
      the protein differing by no more than two conservative amino acid substitutions exclusive of the substitution of a non-naturally-occurring amino acid; the protein substantially retaining all activities of the protein before introduction of the conservative amino acid substitutions.

The invention further includes nucleic acid segments encoding proteins as described above, vectors including the nucleic acid segments, host cells transformed or transfected with the vectors, and methods for producing proteins encoded by the nucleic acid segments.

Additionally, the present invention further includes methods of use. In particular, one method of use of labeled protein molecules according to the present invention is a method of delivering a labeled protein molecule that effects a biological activity to cells, tissue extracellular matrix biomolecule or a biomolecule in the fluid of an individual, wherein the method comprises administering to the individual a labeled protein molecule as described above, wherein the labeled protein molecule is specific for the cells, tissue extracellular matrix biomolecule or fluid biomolecule and wherein the labeled protein molecule effects a biological activity.

Another method of use of labeled proteins according to the present invention is a method of treating or preventing a disease or condition in an individual wherein the disease or condition involves cells, tissue or fluid that expresses a target molecule, the method comprising administering to the individual a therapeutically effective amount of a labeled protein molecule as described above, wherein the labeled protein molecule is specific for the target molecule and wherein the labeled protein molecule effects a biological activity effective against the disease or condition.

Yet another method of use is a method of imaging cells or tissue in an individual wherein the cells or tissue being imaged expresses a molecule bound by the targeting module of a labeled protein according to the present invention, the method comprising the steps of:
(1) administering to the individual a labeled protein according to the present invention as described above; and
(2) detecting the labeled protein bound to the molecule bound to the targeting module.

### BRIEF DESCRIPTION OF THE DRAWINGS

The following invention will become better understood with reference to the specification, appended claims, and accompanying drawings, where:

FIG. 1 is a schematic depiction of a reaction usable to label protein molecules according to the present invention involving the reaction of a hydroxylamine-containing reactive molecule incorporated in a targeting molecule with the amino-terminal amino acid of the protein to be labeled that has, or is modified to contain, an aldehyde-containing side chain.

FIG. 2 is a schematic depiction of a suitable linker used as part of a targeting molecule according to the present invention.

FIG. 3 shows various embodiments of the connecting chain (X) portion of the linker as depicted in FIG. 1.

FIG. 4 is a preferred linker used as part of a targeting molecule according to the present invention.

FIG. 5 is an alternative showing diketo linker reactive groups (Z) and other linker reactive groups, including hydroxylamine and hydrazine.

FIG. 6 shows the structures of other preferred linker reactive groups.

FIG. 7 shows an arrangement in which there are two targeting modules attached to the linker, and the targeting modules are identical.

FIG. 8 shows an arrangement in which there are two targeting modules attached to the linker, and the targeting modules are different.

FIG. 9 shows an arrangement in which there are two targeting module-connecting chain structures in the labeled protein.

FIG. 10 is an example of a unbranched linker.

FIG. 11 is an example of a branched linker.

FIG. 12a is a depiction of a two-step construction of a labeled protein molecule including an Fc region. First, the aldehyde-containing Fc protein is reacted with a hydroxylamine bearing an azide functionality to provide an azide-Fc. The azide-Fc can then be reacted with a wide variety of targeting molecules including a targeting module, a linker, and a reactive group wherein the reactive group includes an alkyne. A copper (I)-catalyzed azide-alkyne [3+2] cycloaddition reaction then produces the labeled protein molecule including the Fc region. Notice that the azide-Fc could also be prepared by translational incorporation of a non-naturally-occurring amino acid bearing a reactive azide group. FIG. 12b is a depiction of an alternative two-step construction of a labeled protein molecule including an Fc region. First, the aldehyde-containing Fc protein is reacted with a bifunctional molecule with two H₂N—O— groups separated by a hydrocarbyl spacer; the product is then reacted further with a diketone.

### DETAILED DESCRIPTION OF THE INVENTION

Definitions

As used herein, the term “nucleic acid,” “nucleic acid sequence,” “polynucleotide,” or similar terms, refers to a deoxyribonucleotide or ribonucleotide oligonucleotide or polynucleotide, including single- or double-stranded forms, and coding or non-coding (e.g., “antisense”) forms. The term encompasses nucleic acids containing known analogues of natural nucleotides. The term also encompasses nucleic acids including modified or substituted bases as long as the modified or substituted bases interfere neither with the Watson-Crick binding of complementary nucleotides or with the binding of the nucleotide sequence by proteins that bind specifically, such as zinc finger proteins. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos.6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzyiphosphonate linkages (see, e.g., U.S. Pat. No. 5,532,226; Samstag (1996) Antisense Nucleic Acid Drug Dev 6:153-156).

As used herein, the term “operatively linked” means that elements of a polypeptide or polynucleotide, for example, are linked such that each performs or functions as intended. For example, an element that regulates expression, such as a promoter, operator, or enhancer, can be operatively linked to the nucleotide sequence whose expression is to be regulated. Linkage between and among elements may be direct or indirect, such as via a linker. The elements are not necessarily adjacent.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. Watson et al. Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) structure and/or activity, e.g., antibody activity, enzymatic activity, or receptor activity. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or lie; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gin); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations” when the three-dimensional structure and the function of the protein to be delivered are conserved by such a variation.

As used herein, the term “expression vector” refers to a plasmid, virus, phagemid, or other vehicle known in the art that has been manipulated by insertion or incorporation of heterologous DNA, such as nucleic acid encoding the fusion proteins herein or expression cassettes provided herein. Such expression vectors typically contain a promoter sequence for efficient transcription of the inserted nucleic acid in a cell. The expression vector typically contains an origin of replication, a promoter, as well as specific genes that permit phenotypic selection of transformed cells.

As used herein, the term “host cells” refers to cells in which a vector can be propagated and its DNA expressed. The term also includes any progeny of the subject host cell. It is understood that all progeny may not be identical to the parental cell since there may be mutations that occur during replication. Such progeny are included when the term “host cell” is used. Methods of stable transfer where the foreign DNA is continuously maintained in the host are known in the art.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors”. Also included are vectors that allow cloning of cDNA (complementary DNA) from mRNAs produced using reverse transcriptase.

As used herein, a gene refers to a nucleic acid molecule whose nucleotide sequence encodes an RNA or polypeptide. A gene can be either RNA or DNA. Genes may include regions preceding and following the coding region (leader and trailer) as well as intervening sequences (introns) between individual coding segments (exons).

As used herein, the term “isolated” with reference to a nucleic acid molecule or polypeptide or other biomolecule means that the nucleic acid or polypeptide has been separated from the genetic environment from which the polypeptide or nucleic acid were obtained. It may also mean that the biomolecule has ben altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide”or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” is meant that the nucleic acid is free of the coding sequences of those genes that, in a naturally-occurring genome immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

“Isolated” or “purified” as those terms are used to refer to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. Particularly for proteins, the procedures may include for example, but are not limited to, ammonium sulfate fractionation, gel filtration, ion exchange change chromatography, affinity chromatography, density gradient centrifugation, electrofocusing, chromatofocusing, and electrophoresis.

A preparation of DNA or protein that is “substantially pure” or “isolated”should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

I. Labeling Methods

One embodiment of the invention is a method for labeling a protein molecule that includes therein the Fc portion of an antibody molecule comprising the steps of:
(1) providing a protein molecule that includes therein the Fc portion of an antibody molecule, the molecule having an amino-terminal serine residue;
(2) oxidizing the amino-terminal serine residue to an aldehyde group; and
(3) reacting the protein molecule with a targeting molecule including therein a moiety reactive with an aldehyde to produce a labeled protein molecule such that the targeting molecule solely directs the targeting of the labeled protein molecule to a target that is a soluble molecule or a cell-surface molecule.

In methods according to the present invention, the labeling of the protein molecule does not occur at the antigen-binding site of the protein molecule in the event that the protein molecule is an intact antibody or a derivative of an intact antibody molecule that is capable of specifically binding an antigen; such labeling is expressly excluded for all methods according to the present invention and for all resulting labeled protein molecules according to the present invention. Additionally, in methods according to the present invention, the labeling of the protein molecule does not occur in framework region 3 of an antibody, more specifically at Kabat residue 93 of the heavy chain of the antibody.

Typically, the moiety reactive with an aldehyde is a hydrazine or other molecule reactive with an aldehyde, such as a hydroxylamine.

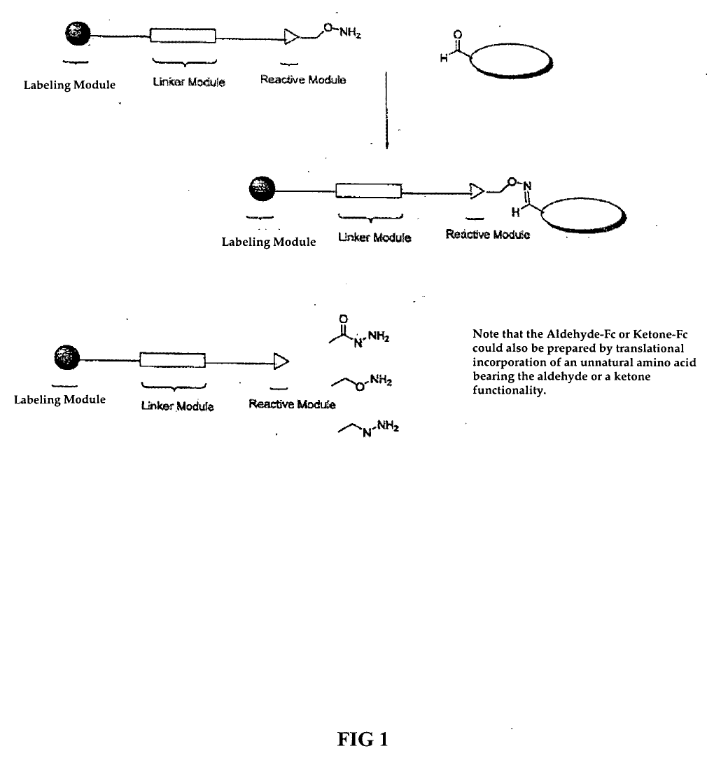

The reaction between the protein molecule and the molecule including therein a moiety reactive with an aldehyde typically is performed in aqueous conditions at a pH of from about 6 to about 10. When the molecule including therein a moiety reactive with an aldehyde is a hydroxylamine, the product is an oxime of structure R₁—O—N═CH—R₂, wherein R₂ is the remainder of the protein molecule and R₁ is the remainder of the targeting molecule. This reaction is depicted schematically in FIG. 1. FIG. 1 is a schematic depiction of a reaction usable to label protein molecules according to the present invention involving the reaction of a hydroxylamine-containing reactive moiety incorporated in a targeting molecule with the amino-terminal amino acid of the protein to be labeled that has, or is modified to contain, an aldehyde-containing side chain or a ketone-containing side chain. As discussed below, in another alternative, the amino-terminal residue, instead of being a serine that is oxidized to an aldehyde, is incorporated as a non-naturally-occurring amino acid that contains a carbonyl group. This alternative is also depicted in FIG. 1.

In one alternative, the amino-terminal serine is oxidized to an aldehyde function by oxidation with periodate to a glyoxylyl residue, as described in K. F. Geoghegan & J. G. Stroh, “Site-Directed Conjugation of Nonpeptide Groups to Peptides and Proteins via Periodate Oxidation of a 2-Amino Alcohol. Application to Modification at N-Terminal Serine,” Bioconjugate Chem. 3:138-148 (1992), and in K. F. Geoghegan et al., “Site-Directed Double Fluorescent Tagging of Human Renin and Collagenase (MMP-1) Substrate Peptides Using the Periodate Oxidation of N-Terminal Serine. An Apparently General Strategy for Provision of Energy-Transfer Substrates for Proteases,” Bioconiugate Chem. 4: 537-644 (1993), both incorporated herein by this reference. Typically, the oxidation occurs at a pH of about 7.

The protein molecule is typically an intact antibody molecule or the Fc domain of an antibody molecule, subject to the provisos above with respect to the position of labeling of the labeled protein molecule by the targeting module. Alternatively, the protein molecule is a protein molecule that includes the Fc domain of an antibody molecule plus additional amino acid sequences. In either case, the protein molecule incorporates the C-terminal portion of the heavy chain of an antibody molecule. However, the protein molecule can be any member of the Ig superfamily that has a region substantially homologous to an Fc domain This includes, but is not limited to, TCR β, and MHC Class I and II proteins. Other protein molecules can be used for labeling, again subject to the provisos above with respect to the position of labeling of the labeled protein molecule by the targeting module.

The Fc regions of protein molecules used in labeling methods according to the present invention can be modified to have increased potency, either by mutagenesis of the amino acid sequence or by changing the pattern of glycosylation. Methods for these modifications are described in T. Shinkawa et al., “The Absence of Fucose but Not the Presence of Galactose or Bisecting N-Acetylglucosamine of Human IgG1 Complex-Type Oligosaccharides Shows the Critical Role of Enhancing Antibody-Dependent Cellular Cytotoxicity,” J. Biol. Chem. 278: 3466-3473 (2003) and L. G. Presta et al., “Engineering Therapeutic Antibodies for Improved Function,” Biochem. Soc. Trans. 30: 487-490 (2002), incorporated herein by this reference.

Alternatively, the protein labeled in methods according to the invention can include various portions of the Fc fragment, such as C_{H}3 alone or C_{H}1 —C_{H}2—C_{H}3 paired with C_{L}; in the latter case, the constant regions of the heavy and light chains are held together with interchain disulfide bonds. In some applications, it can be desirable to include the hinge region, so that the protein labeled according to methods of the present invention can include constructs of the form: hinge-C_{H}2—C_{H}3; C_{H}1-hinge-C_{H}2—C_{H}3 paired with C_{L}; or hinge-C_{H}3 in addition to the ones described above, or similar constructs lacking the hinge region.

In another alternative, other proteins, peptides, or domains from other proteins, can be fused to the carboxyl terminus of the Fc. These proteins can include, but are not limited to, a cytokine like IL-2, or even another antibody fragment like a scFv wherein the N-terminus of the Fc is still used for covalent linkage to a targeting molecule. These proteins can also include enzymes or receptors, as well as peptides such as a polyhistidine or a FLAG purification tag.

Typically the protein molecule is produced by site-directed mutagenesis of a naturally-occurring protein molecule, such that the amino-terminal residue is mutated to a serine residue or other reactive residue as described further below, such as a reactive cysteine residue. Methods for performing site-directed mutagenesis are well-known in the art and need not be described further in detail; they are described in J. Sambrook & D. W. Russell, “Molecular Cloning: A Laboratory Manual” (3^{rd} ed., Cold Spring Harbor Laboratory Press, Cold Spring Harbor, N.Y. 2001), v.2, ch. 13, incorporated herein by this reference. These methods include, but are not necessarily limited to, oligonucleotide-directed mutagenesis and PCR-mediated site-directed mutagenesis.

As detailed below, the protein molecule can be produced by transforming or transfecting a suitable host cell with a vector including therein a nucleotide sequence encoding the protein molecule.

In one preferred embodiment, the targeting molecule comprises: (1) a targeting module; (2) a linker covalently linked to the targeting module; and (3) a reactive module covalently linked to the linker, the reactive module including therein a hydroxylamine moiety or derivative thereof. As described above, other moieties reactive with the aldehyde group can be used instead of the hydroxylamine moiety, such as a hydrazine or a hydrazide.

In another alternative, the targeting molecule comprises: (1) a targeting module; and (2) a reactive module covalently linked to the linker, the reactive module including therein a hydroxylamine moiety or derivative thereof, or other moiety reactive with the aldehyde group, In this alternative, the linker is omitted.

The targeting module can be any moiety that binds to and targets a particular biomolecule, e.g., one located on a cell such as on the surface of a cell, tissue (e.g. extracellular matrix), fluid, organism, or subset thereof. The biomolecule is typically a protein or peptide, but could be a carbohydrate, a nucleic acid, a glycoprotein, a lipid, a glycolipid, or another molecule that could be targeted, The targeting reaction can be used for either diagnostic purposes or for therapy. In some alternatives, the targeting module is either detectable or can yield a detectable product, either directly or through a secondary reaction.

In one preferred embodiment, the molecule to be targeted is an integrin, and the targeting module is an integrin antagonist or a peptide such as an RGD type peptides that binds an integrin. Examples of suitable targeting modules for targeting integrins are those described in C. Rader et al., “Programmed Monoclonal Antibodies for Cancer Therapy: Adaptor lmmunotherapy Based on a Covalent Antibody Catalyst,” Proc. Nat. Acad. Sci. USA, 100:5396-5400 (2003) and in L.-S. Li et al., “Chemical Adaptor Immunotherapy: Design, Synthesis, and Evaluation of Novel Integrin-Targeting Devices,” J. Med. Chem. 47:5630-40 (2004), both incorporated herein by this reference. These molecules can be modified by including a hydroxylamine moiety instead of the ketone moiety as described in these references to enable them to be conjugated to the aldehyde-containing amino acid as described above.

Suitable targeting modules include, but are not limited to those described in U.S. Patent Application Publication No. 2003/0129188 by Barbas et al., in U.S. Patent Application Publication No. 2003/0190676 by Barbas et al., and in U.S. Patent Application Publication No. 2003/0175921 by Barbas et al., all incorporated herein by this reference.

In general, the targeting module is incorporated into the labeled protein molecule in a manner that does not affect its binding specificity for the target, such as by sufficiently distancing the targeting agent from the remainder of the labeled protein molecule, such as the Fc portion of an antibody, so that it can bind its target without steric hindrance by the Fc portion of the antibody.

“Targeting module” as used herein refers to a moiety that recognizes, binds or adheres to a target moiety of a target molecule located for example on a cell, tissue (e.g. extracellular matrix), fluid, organism, or subset thereof A targeting module and its target molecule represent a binding pair of molecules, which interact with each other through any of a variety of molecular forces including, for example, ionic, covalent, hydrophobic, van der Waals, and hydrogen bonding, so that the pair have the property of binding specifically to each other. Specific binding means that the binding pair exhibit binding with each other under conditions where they do not significantly bind to another molecule. Examples of binding pairs are biotin-avidin, hormone-receptor, receptor-ligand, enzyme-substrate, IgG-protein A, antigen-antibody, and the like. The targeting agent and its cognate target molecule exhibit a significant association for each other. This association may be evaluated by determining an equilibrium association constant (or binding constant) according to methods well known in the art. Affinity is calculated as K_{d}=k_{off}/kₒₙ (k_{off} is the dissociation rate constant, kₒₙ is the association rate constant and K_{d} is the equilibrium constant.

Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and [0045] n=number of ligand binding sites per receptor molecule.

By graphical analysis, r/c is plotted on the Y-axis versus r on the X-axis thus producing a Scatchard plot. The affinity is the negative slope of the line. The constant off can be determined by competing bound labeled ligand with unlabeled excess ligand (see, e.g., U.S. Pat. No. 6,316,409). The affinity of a targeting module or targeting molecule for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter.

Targeting modules include, but are not limited to, small molecule organic compounds of 5,000 daltons or less such as drugs, proteins, peptides, peptidomimetics, glycoproteins, proteoglycans, lipids, glycolipids, phospholipids, lipopolysaccharide, nucleic acids, proteoglycans, carbohydrates, and the like. Targeting modules may include well known therapeutic compounds including anti-neoplastic agents. Anti-neoplastic targeting agents may include paclitaxel, daunorubicin, carminomycin, 4′-epiadriamycin, 4-demethoxy-daunomycin, 11-deoxydaunorubicin, 13-deoxydaunorubicin, adriamycin-14-benzoate, adriamycin-14-octanoate, adriamycin-14-naphthalene acetate, vinblastine, vincristine, mitomycin C, N-methyl mitomycin C, bleomycin A₂, dideazatetrahydrofolic acid, aminopterin, methotrexate, colchicine and cisplatin, and the like. Anti-microbial agents include aminoglycosides including gentamicin, antiviral compounds such as rifampicin, 3′-azido-3′-deoxythymidine (AZT) and acylovir, antifungal agents such as azoles including fluconazole, macrolides such as amphotericin B, and candicidin, anti-parasitic compounds such as antimonials, and the like. Hormone targeting agents include toxins such as diphtheria toxin, cytokines such as CSF, GSF, GMCSF, TNF, erythropoietin, immunomodulators or cytokines such as the interferons or interleukins, a neuropeptide, reproductive hormone such as HGH, FSH, or LH, thyroid hormone, neurotransmitters such as acetylcholine, and hormone receptors such as the estrogen receptor.

The targeting molecule, including the targeting module and the linker, preferably is at least about 300 daltons in size, and preferably may be at least about 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500 or even 5,000 daltons in size, with even larger sizes possible.

Suitable targeting modules in targeting molecules of the invention can be a protein or peptide. “Polypeptide”, “peptide,” and “protein” are used interchangeably to refer to a polymer of amino acid residues. As used herein, these terms apply to amino acid polymers in which one or more amino acid residue is an artificial chemical analogue of a corresponding naturally occurring amino acid. These terms also apply to naturally occurring amino acid polymers. Amino acids can be in the L or D form as long as the binding function of the peptide is maintained. Peptides can be of variable length, but are generally between about 4 and 200 amino acids in length. Peptides may be cyclic, having an intramolecular bond between two non-adjacent amino acids within the peptide, e.g., backbone to backbone, side-chain to backbone and side-chain to side-chain cyclization. Cyclic peptides can be prepared by methods well known in the art. See e.g., U.S. Pat. No. 6,013,625.

Protein or peptide targeting modules that exhibit binding activity for a target molecule are well known in the art. For example, a targeting module may be a viral peptide cell fusion inhibitor. This may include the T-20 HIV-1 gp41 fusion inhibitor which targets fusion receptors on HIV infected cells (for T-20, see U.S. Pat. Nos. 6,281,331 and 6,015,881 to Kang et al.; Nagashima et al. J. Infectious Diseases 183:1121, 2001; for other HIV inhibitors see U.S. Pat. No. 6,020,459 to Barney and WO 0151 673A2 to Jeffs et al), RSV cell fusion inhibitors (see WO 0164013A2 to Antczak and McKimm-Breschkin, Curr. Opin. Invest. Drugs 1:425-427, 2000 (VP-14637)), pneumovirus genus cell fusion inhibitors (see WO 9938508A1 by Nitz et al.), and the like. Targeting modules also include peptide hormones or peptide hormone analogues such as LHRH, bombesin/gastrin releasing peptide, somatostatin (e.g., RC-121 octapeptide), and the like, which may be used to target any of a variety of cancers, e.g., ovarian, mammary, prostate small cell of the lung, colorectal, gastric, and pancreatic. See, e.g., Schally et al., Eur. J. Endocrinology, 141:1-14, 1999.

Peptide targeting modules suitable for use in labeled proteins according to the invention also may be identified using in vivo targeting of phage libraries that display a random library of peptide sequences (see, e.g., Arap et al., Nature Medicine, 2002 8(2):121-7; Arap et al., Proc. Nati. Acad. Sci. USA 2002 99(3):1527-1531; Trepel et al. Curr. Opin. Chem. Biol. 2002 6(3):399-404).

In some embodiments, the targeting module is specific for an integrin. Integrins are heterodimeric transmembrane glycoprotein complexes that function in cellular adhesion events and signal transduction processes. Integrin αᵥβ₃ is expressed on numerous cells and has been shown to mediate several biologically relevant processes, including adhesion of osteoclasts to bone matrix, migration of vascular smooth muscle cells, and angiogenesis. Integrin αᵥβ₃ antagonists likely have use in the treatment of several human diseases, including diseases involving neovascularization, such as rheumatoid arthritis, cancer, and ocular diseases.

Suitable targeting agents for integrins include RGD peptides or peptidomimetics or non-RGD peptides or peptidomimetics. As used herein, reference to “rg-Gly-Asp peptide” or “RGD peptide” is intended to refer to a peptide having one or more Arg-Gly-Asp containing sequence which may function as a binding site for a receptor of the “Arg-Gly-Asp family of receptors”, e.g., an integrin. Integrins, which comprise an alpha and a beta subunit, include numerous types including, α₁β₁, α₂β₁, α₃β₁, α₄β₁, α₅β₁, α₆β₁, α₇β₁, α₈β₁, α₉β₁, α₆β₄, α₄β₇, α_{D}β₂, α_{L}β₂, α_{M}β₂, αᵥβ₁, αᵥβ₃, αᵥβ₃, αᵥβ₆, αᵥβ₈, αₓβ₂, α_{IIb}β₃, α_{IELb}β₇, and the like. The sequence RGD is present in several matrix proteins and is the target for cell binding to matrix by integrins. Platelets contain a large amount of RGD-cell surface receptors of the protein GP II_{b}/IIIₐ, which is primarily responsible, through interaction with other platelets and with the endothelial surface of injured blood vessels, for the development of coronary artery thrombosis. The term RGD peptide also includes amino acids that are functional equivalents (e.g., RLD or KGD) thereof provided they interact with the same RGD receptor. Peptides containing RGD sequences can be synthesized from amino acids by means well known in the art, using, for example, an automated peptide synthesizer, such as those manufactured by Applied Biosystems, Inc., Foster City, Calif.

As used herein, “non-RGD” peptide refers to a peptide that is an antagonist or agonist of integrin binding to its ligand (e.g. fibronectin, vitronectin, laminin, collagen etc.) but does not involve an RGD binding site. Non-RGD integrin peptides are known for α_{V}β₃ (see, e.g., U.S. Pat. Nos. 5,767,071 and 5,780,426) as well as for other integrins such as α₄β₁ (VLA-4), . α₄β₇ (see, e.g., U.S. Pat. No. 6,365,619; Chang et al., Bioorganic & Medicinal Chem Lett, 12:159-163 (2002); Lin et al., Bioorganic & Medicinal Chem Lett, 12:133-136 (2002)), and the like.

An integrin targeting module may be a peptidomimetic agonist or antagonist, which preferably is a peptidomimetic agonist or antagonist of an RGD peptide or non-RGD peptide. As used herein, the term “peptidomimetic” is a compound containing non-peptidic structural elements that are capable of mimicking or antagonizing the biological action(s) of a natural parent peptide. A peptidomimetic of an RGD peptide is an organic molecule that retains similar peptide chain pharmacophore groups of the RGD amino acid sequence but lacks amino acids or peptide bonds in the binding site sequence. Likewise, a peptidomimetic of a non-RGD peptide is an organic molecule that retains similar peptide chain pharmacophore groups of the non-RGD binding site sequence but lacks amino acids or peptide bonds in the binding site sequence. A “pharmacophore” is a particular three-dimensional arrangement of functional groups that are required for a compound to produce a particular response or have a desired activity. The term “RGD peptidomimetic” is intended to refer to a compound that comprises a molecule containing the RGD pharmacophores supported by an organicinon-peptide structure. It is understood that an RGD peptidomimetic (or non-RGD peptidomimetic) may be part of a larger molecule that itself includes conventional or modified amino acids linked by peptide bonds.

RGD peptidomimetics are well known in the art, and have been described with respect to integrins such as GPII_{b}/IIIₐ, αᵥβ₃ and αᵥβ₅ (See, e.g., Miller et al., J. Med. Chem. 2000, 43:22-26; and International Pat. Publications WO 0110867, WO 9915178, WO 9915170, WO 9815278, WO 9814192, WO 0035887, WO 9906049, WO 9724119 and WO 9600730; see also Kumar et al., Cancer Res. 61:2232-2238 (2000)). Many such compounds are specific for more than one integrin. RGD peptidomimetics are generally based on a core or template (also referred to as “fibrinogen receptor antagonist template”), to which are linked by way of spacers to an acidic group at one end and a basic group at the other end of the core. The acidic group is generally a carboxylic acid functionality while the basic group is generally a N-containing moiety such as an amidine or guanidine, Typically, the core structure adds a form of rigid spacing between the acidic moiety and the basic nitrogen moiety, and contains one or more ring structures (e.g., pyridine, indazole, etc.) or amide bonds for this purpose. For a fibrinogen receptor antagonist, generally, about twelve to fifteen, more preferably thirteen or fourteen, intervening covalent bonds are present (via the shortest intramolecular path) between the acidic group of the RGD peptidomimetic and a nitrogen of the basic group. The number of intervening covalent bonds between the acidic and basic moiety is generally shorter, two to five, preferably three or four, for a vitronectin receptor antagonist. The particular core may be chosen to obtain the proper spacing between the acidic moiety of the fibrinogen antagonist template and the nitrogen atom of the pyridine. Generally, a fibrinogen antagonist will have an intramolecular distance of about 16 Å (1.6 nm) between the acidic moiety (e.g., the atom which gives up the proton or accepts the electron pair) and the basic moiety (e.g., which accepts a proton or donates an electron pair), while a vitronectin antagonist will have about 14 Å (1.4 nm) between the respective acidic and basic centers. Further description for converting from a fibrinogen receptor mimetic to a vitronectin receptor mimetic can be found in U.S. Pat. No. 6,159,964.

The peptidomimetic RGD core can comprise a 5-11 membered aromatic or nonaromatic mono- or polycyclic ring system containing 0 to 6 double bonds, and containing 0 to 6 heteroatoms chosen from N, O and S. The ring system may be unsubstituted or may be substituted on a carbon or nitrogen atom. Preferred core structures with suitable substituents useful for vitronectin binding include monocyclic and bicyclic groups, such as benzazapine described in WO 98/14192, benzdiazapine described in U.S. Pat. No. 6,239,168, and fused tricyclics described in U.S. Pat No. 6,008,213.

U.S. Pat. No. 6,159,964 contains an extensive list of references in Table 1 of that document which disclose RGD peptidomimetic cores structures (referred to as fibrinogen templates) which can be used for prepraring RGD peptidomimetics. Preferred vitronectin RGD and fibronectin RGD peptidomimetics are disclosed in U.S. Pat. Nos. 6,335,330; 5,977,101; 6,088,213; 6,069,158; 6,191,304; 6,239,138; 6,159,964; 6,117,910; 6,117,866; 6,008,214; 6,127,359; 5,939,412; 5,693,636; 6,403,578; 6,387,895; 6,268,378; 6,218,387; 6,207,663; 6,011,045; 5,990,145; 6,399,620; 6,322,770; 6,017,925; 5,981,546; 5,952,341; 6,413,955; 6,340,679; 6,313,119; 6,268,378; 6,211,184; 6,066,648; 5,843,906; 6,251,944; 5,952,381; 5,852,210; 5,811,441; 6,114,328; 5,849,736; 5,446,056; 5,756,441; 6,028,087; 6,037,343; 5,795,893; 5,726,192; 5,741,804; 5,470,849; 6,319,937; 6,172,256; 5,773,644; 6,028,223; 6,232, 308; 6,322,770; 5,760,028.

Exemplary RGD peptidomimetic integrin targeting agents, such as those shown as compounds 1, 2, and 3 in U.S. Patent Application Publication No. 2003/0129188 by Barbas et al., can be used for preparing an intregrin targeting module as part of a labeled protein according to the present invention. These compounds are modified or attached to a linker such that they have a moiety capable of reacting with the aidehyde-containing amino acid of the protein molecule as described above. In the three compounds, the linker is attached as indicated to the nitrogen of the seven membered ring. Other RGD peptidomimetic integrin targeting agents include compound 33 as shown in U.S. Patent Application Publication No. 2003/0129188 by Barbas et al., wherein P and L are carbon or nitrogen. The linker may be R1 or R2 while the R3 group includes a basic group such as an —NH group. In some embodiments, the R3 group is as shown in compounds 1, 2, or 33 of U.S. Patent Application Publication No. 2003/0129188 by Barbas et al. In some embodiments, the R3 group includes a heterocyclic group such a benzimidazole, imidazole, pyridine group, or the like. In some such embodiments, the R3 group is a alkoxy group, such as a propoxy group or the like, that is substituted with a heterocyclic group that is substituted with an alkylamine group, such as a methylamino group or the like, whereas in other embodiments, the R3 group is an alkoxy group, such as a propoxy group or the like, substituted with a heterocyclylamino group, such as with a pyridinylamino group or the like such as a 2-pyridinylamino group. In other embodiments R3 is a group of formula —C(═O)Rb where Rb is selected from —N(alkyl)-alkyl-heterocyclic groups such as —N(Me)—CH₂-benzimidazole groups and the like.

Other exemplary integrin peptidomimetic targeting modules and a peptide targeting module are shown in FIG. 1 of U.S. Patent Application Publication No. 2003/0129188 by Barbas et al. The linker may be any of R₁, R₂, R₃, while R₄ may be a linker or a hydrolyzable group such as alkyl, alkenyl, alkynyl, oxoalkyl, oxoalkenyl, oxoalkynyl, aminoalkyl, aminoalkenyl, aminoalkynyl, sulfoalkyl, sulfoalkenyl, or sulfoalkynyl group, phosphoalkyl, phosphoalkenyl, phosphoalkynyl group, and the like, as described in U.S. Patent Application Publication No. 2003/0129188 by Barbas et al. One of skill in the art will readily appreciate that other integrin agonist and antagonist mimetics can also be used in targeting modules of the present invention.

The target molecule to which the targeting module binds is preferably a non-immunoglobulin molecule or is an immunoglobulin molecule where the target moiety is outside the immunoglobulin combining site. It is not intended to exclude from the inventive compounds those targeting agents that function as antigens and, therefore, bind to an immunoglobulin combining site; this binding is to be distinguished from the covalent binding that generates the labeled molecule, as described above. Such targeting modules are included herein provided the targeting modules also bind to a non-immunoglobulin molecule and/or a target moiety located outside the combining site of an immunoglobulin molecule. In general, the target molecule can be any type of molecule including organic, inorganic, protein, lipid, carbohydrate, nucleic acid and the like.

Still other targeting molecules are within the scope of the invention. These include the modified T-20 peptide having the amino acid sequence N-Acetyl-YTSLIHSLIEESQNQQEKNEQELLELDKWASLWNWFC (SEQ ID NO: 1). This peptide is a derivative of the peptide T-20, N-Acetyl-YTSLIHSLIEESQNQQEKNEQELLELDKWASLWNWF (SEQ ID NO: 2), with an additional N-terminal cysteine. T-20 is a synthetic peptide corresponding to a region of the transmembrane subunit of the HIV-1 envelope protein, and that blocks cell fusion and viral entry at concentrations of less than 2 ng/ml in vitro. When administered intravenously, T-20 (monotherapy), the peptide decreases plasma HIV RNA levels demonstrating that viral entry can be successfully blocked in vivo. Administration of T-20 provides potent inhibition of HIV replication comparable to anti-retroviral regimens approved at present (Kilby et al., Nat Med., 1998, 4(11):1302-7). This peptide drug suffers from a short half-life in vivo of approximately 2 hrs. The thiol-labeled peptide is suitable for use as a targeting module and can be used to inhibit HIV-1 entry and infection, as described in Example 8 of U.S. Patent Application Publication No. 2003/0129188 by Barbas et al., incorporated herein by this reference. In addition to peptides that target the envelope proteins of HIV-1, a number of small-molecules that bind the envelope proteins have been described. For example, the betulinic acid derivative IC9564 is a potent anti-human immunodeficiency virus (anti-HIV) compound that can inhibit both HIV primary isolates and laboratory-adapted strains. Evidence suggests that HIV-1 gp120 plays a key role in the anti-HIV-1 activity of IC9564 (Holz-Smith et al., Antimicrob Agents Chemother., 2001, 45(1):60-6.) Preparing an antibody targeting compound in which IC9564 is the targeting agent is expected to have increased activity over IC9564 itself by increasing valency, half-life, and by directing immune killing of HIV-1 infected cells based on the constant region of the antibody chosen. Similarly, recent X-ray crystallographic determination of the HIV-1 envelope glycoprotein gp4l core structure opened up a new avenue to discover antiviral agents for chemotherapy of HIV-1 infection and AIDS. Compounds with the best fit for docking into the hydrophobic cavity within the gp41 core and with maximum possible interactions with the target site can also be improved by addition of a diketone arm and covalent linkage to an antibody. Several compounds of this class have been identified (Debnath et al., J Med Chem., 1999, 42(17):3203-9). These peptides and their derivatives can be used as targeting modules in the same manner as cysteine-labeled T-20.

The target molecule is preferably a biomolecule such as a protein, carbohydrate, lipid or nucleic acid. The target molecule can be associated with a cell (“cell surface expressed”), or other particle (“particle surface expressed”) such as a virus, or may be extracellular such as a molecule in serum or other fluid. If associated with a cell or particle, the target molecule is preferably expressed on the surface of the cell or particle in a manner that allows the targeting agent of the targeting compound to make contact with the surface receptor from the fluid phase of the body.

In some preferred embodiments, the target molecule is predominantly or exclusively associated with a pathological condition or diseased cell, tissue or fluid. Thus, the targeting molecule of a labeled protein according to the present invention can be used to deliver the targeting molecule to a diseased tissue by targeting the cell, an extracellular matrix biomolecule or a fluid biomolecule. Exemplary target molecules disclosed hereinafter in the Examples of U.S. Patent Application Publication No. 2003/0129188 by Barbas et al. include integrins (Example 1), cytokine receptors (Examples 2, 3 and 7), cytokines (Example 4), vitamin receptors (Example 5), cell surface enzymes (Example 6), and HIV-1 virus and HIV-1 virus infected cells (Examples 8 and 11), and the like.

In other preferred embodiments, the target molecule is associated with an infectious agent and is expressed on the surface of a microbial cell or on the surface of a viral particle. As such, labeled proteins according to the present invention in which the targeting module can bind to the cell surface expressed or particle expressed infectious agent can be used as an anti-microbial, by targeting microbial agents inside the body or on the surface (e.g., skin) of an individual. In the latter case, the invention compound can be applied topically.

Antibody targeting modules or targeting molecules specific for a microbial target molecule also can be used as an anti-microbial agent in vitro. Accordingly, a method of reducing the infectivity of microbial cells or viral particles present on a surface is provided. Some methods include contacting the surface of a microbial cell or viral particle with an effective amount of the invention targeting compound. The targeting compound in such methods includes a targeting agent specific for a receptor on the microbial cell or virus particle. Applicable surfaces are any surfaces in vitro such as a counter top, condom, and the like.

Another preferred target molecule for targeting molecules or targeting modules of the invention is prostate specific antigen (PSA), a serine protease that has been implicated in a variety of disease states including prostate cancer, breast cancer and bone metastasis. Specific inhibitors of PSA which bind to the active site of PSA are known. See Adlington et al., J. Med. Chem., 2001, 44:1491-1508 and WO 98/25895 to Anderson. A specific inhibitor of PST is shown in U.S. Patent Application Publication No. 2003/0129188 by Barbas et al. as compound 34.

A targeting module or targeting molecule, in addition to its ability to bind a target molecule, may be characterized in having one or more biological activities, each activity characterized as a detectable biological effect on the functioning of a cell organ or organism. Thus, in addition to being a targeting module, such compounds can be considered biological agents. For example, the integrin targeting modules shown as compounds 1, 2, 3 and 33 in U.S. Patent Application Publication No. 2003/0129188 by Barbas et al., or derivatives of these molecules possessing a hydroxylamine group or other group capable of reacting with an aldehyde-containing amino acid as described above, above not only target an integrin, but have integrin antagonist biological activity. In some embodiments, however, a targeting module may be a pure binding agent without biological activity or may possess agonist activity; TPO peptides are an example.

Particular targeting modules or targeting molecules may or may not possess biological activity depending on the context of their use.

Biological agent functional components include, but are not limited to, small molecule drugs (a pharmaceutical organic compound of about 5,000 daltons or less), organic molecules, proteins, peptides, peptidomimetics, glycoproteins, proteoglycans, lipids, glycolipids, phospholipids, lipopolysaccharides, nucleic acids, proteoglycans, carbohydrates, and the like. Biological agents may be anti-neoplastic, anti-microbial, a hormone, an effector, and the like. Such compounds include well known therapeutic compounds such as the anti-neoplastic agents paclitaxel, daunorubicin, carminomycin, 4′-epiadriamycin, 4-demethoxy-daunomycin, 11-deoxydaunorubicin, 13-deoxydaunorubicin, adriamycin-14-benzoate, adriamycin-14-octanoate, adriamycin-14-naphthalene acetate, vinblastine, vincristine, mitomycin C, N-methyl mitomycin C, bleomycin A₂, dideazatetrahydrofolic acid, aminopterin, methotrexate, colchicine and cisplatin, and the like. Anti-microbial agents include aminoglycosides including gentamicin, antiviral compounds such as rifampicin, 3′-azido-3′-deoxythymidine (AZT) and acylovir, antifungal agents such as azoles including fluconazole, macrolides such as amphotericin B, and candicidin, anti-parasitic compounds such as antimonials, and the like. Hormones may include toxins such as diphtheria toxin, cytokines such as CSF, GSF, GMCSF, TNF, erythropoietin, immunomodulators or cytokines such as the interferons or interleukins, a neuropeptide, reproductive hormone such as HGH, FSH, or LH, thyroid hormone, neurotransmitters such as acetylcholine, hormone receptors such as the estrogen receptor. Also included are non-steroidal anti-inflammatories such as indomethacin, salicylic acid acetate, ibuprofen, sulindac, piroxicam, and naproxen, and anesthetics or analgesics. Also included are radioisotopes such as those useful for imaging as well as for therapy.

Biological agent functional components for use in the targeting modules or targeting molecules of labeled proteins according to the invention can be naturally occurring or synthetic. Biological agents can be biologically active in their native state, or be biologically inactive or in a latent precursor state and acquire biological or therapeutic activity when a portion of the biological agent is hydrolyzed, cleaved or is otherwise modified. The prodrug can be delivered at the surface of a cell or intracellularly using antibody targeting compounds of the invention where it can then be activated. In this regard, the biological agent can be a “prodrug,” meaning that prodrug molecules capable of being converted to drugs (active therapeutic compounds) by certain chemical or enzymatic modifications of their structure. In the prodrug approach, site-specific drug delivery can be obtained from tissue-specific activation of a prodrug, which is the result of metabolism by an enzyme that is either unique for the tissue or present at a higher concentration (compared with other tissues); thus, it activates the prodrug more efficiently.

In another alternative, the targeting molecule can primarily function as a label for the target; for example, the targeting module can be a fluorescent, chemiluminescent, or bioluminescent molecule. The targeting module can also incorporate a direct label, such as a colloidal gold label. The targeting module can also be any molecule incorporating a detectable radioisotope. As another alternative, the targeting module can be a protein, such as an enzyme that catalyzes a reaction that produces a detectable product. In another alternative, the targeting module can be a protein that is detected by the use of a secondary labeled antibody that specifically binds the targeting module. The product can be detectable colorimetrically, by fluorescence, by chemiluminescence, by bioluminescence, or by its reaction with another molecule. An example is the hydrolytic enzyme β-galactosidase. The targeting module can also be detectable by a biological property, such as drug resistance. Accordingly, the targeting module can be or include a protein such as an enzyme, another antibody or portion thereof, or a receptor, as well as a ligand for a receptor. Receptors can include thrombospondin receptors, such as CD36, as well as VEGF receptors or TNFα receptors. Ligands for receptors can include ligands for thrombospondin receptors, ligands for VEGF receptors, or ligands for TNFα receptors. Therefore, as used herein, the term “targeting module” (without an attached linker) or “targeting molecule” (with an attached linker) are used as described above to include molecules that have targeting or labeling activity as described above, unless otherwise further specified.

In another alternative, the diketone-containing molecules described in U.S. Patent Application Publication No. 2003/0129188 by Barbas et al., in U.S. Patent Application Publication No. 2003/0190676 by Barbas et al., and in U.S. Patent Application Publication No. 2003/0175921 by Barbas et al., all incorporated herein by this reference can be used as targeting molecules by modifying the protein molecule, such as the Fc portion of an antibody molecule, to incorporate a hydrazine moiety.

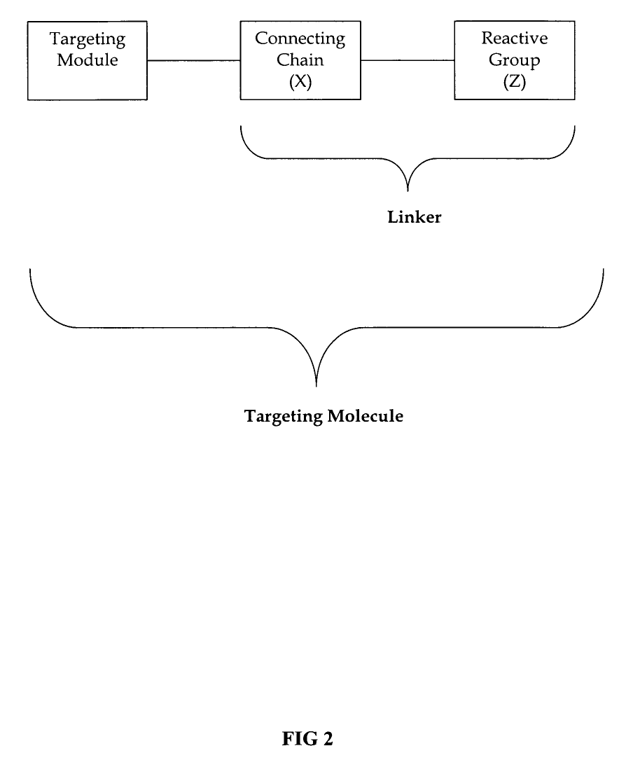

Suitable linkers are described, for example, in U.S. Patent Application Publication No. 2003/0129188 by Barbas et al., in U.S. Patent Application Publication No. 2003/0190676 by Barbas et al., and in U.S. Patent Application Publication No. 2003/0175921 by Barbas et al., all incorporated herein by this reference. In general, the structure of the linker is schematically shown in FIG. 2. The linker typically includes a connecting chain (X) and the reactive group (Z), which is, in this embodiment, a hydroxylamine moiety.

In one embodiment, the linker has the general structure X—Z wherein X is a linear or branched connecting chain of atoms comprising any of C, H, N, O, P, S, Si, F, Cl, Br, and I, or a salt thereof, and comprising a repeating ether unit of between 2-100 units; and Z is a hydroxylamine moiety, in this embodiment, as described above. The linker can be linear or branched and optionally includes one or more carbocyclic or heterocyclic groups. In some embodiments, the linker has a linear stretch of between 5-200 or 10-200 atoms although in other embodiments, longer linker lengths may be used. One or more targeting modules can be linked to X. In some embodiments, where more than one targeting module is linked and a branched linker is used, some of the targeting modules may be linked to different branches of the linker. However, it should be understood that linkers used in the compounds of the invention may have one or more reactive groups and one or more connecting chains and combinations thereof. Connecting chains may branch from another connecting chain.

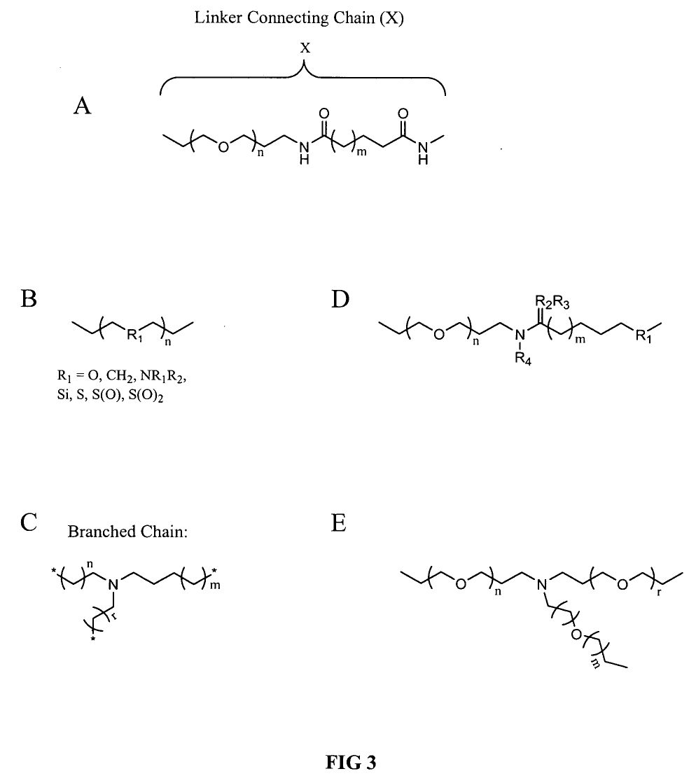

Various embodiments of the connecting chain X portion of the general linker design (FIG. 2) are shown in FIG. 3. As shown, the connecting chain may vary considerably in length with both straight chain and branched chain structures possible.

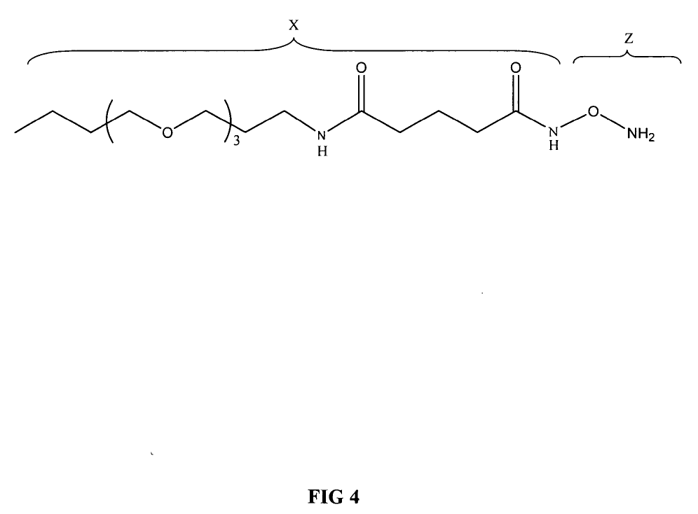

A preferred linker for use in methods and compounds according to the present invention is a linker having the structure shown in FIG. 4 where n is from 1-100 or more and preferably is 1, 2, or 4, and more preferably is 3. In some embodiments, the linker is a repeating polymer such as polyethylene glycol or includes a polyethylene glycol moiety.

An appropriate linker can be chosen to provide sufficient distance between the targeting molecule and the protein molecule, depending on the required interactions of both the targeting molecule and the protein molecule with their ligands. This distance depends on several factors including, for example, the nature of the interactions between the protein and its ligands and the nature of the targeting molecule. Generally, the linker will be between about 5 to 10 Å (0.5 to 1 nm) in length, with a length of 10 Å (1.0 nm) or more being more preferred, although shorter linkers of about 3 Å (0.3 nm) in length may be sufficient if the targeting molecule includes a segment that can function as a part of a linker.

Linker length may also be viewed in terms of the number of linear atoms (cyclic moieties such as aromatic rings and the like to be counted by taking the shortest route). Linker length under this measure is generally about 10 to 200 atoms and more typically about 30 or more atoms, although shorter linkers of two or more atoms may be sufficient in some instances. Generally, linkers with a linear stretch of at least about 9 atoms are sufficient.

Other linker considerations include the effect of the linker on physical or pharmacokinetic properties of the resulting targeting molecule and of the resulting complex between the targeting molecule and the protein. These properties include, but are not limited to, solubility, lipophilicity, hydrophilicity, hydrophobicity, stability (more or less stable as well as planned degradation), rigidity, flexibility, immunogenicity, modulation of binding, chemical compatibility, ability to be incorporated into a micelle or liposome, and the like.

In some embodiments, the connecting chain of the linker includes any atom from the group C, H, N, O, P, S, Si, halogen (F, Cl, Br, I) or a salt thereof. The linker also may include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, or sulfoalkynyl group, phosphoalkyl, phosphoalkenyl, phosphoalkynyl group, as well as a carbocyclic or heterocyclic mono or fused saturated or unsaturated ring structure. Combinations of the above groups and rings may also be present in the linkers of the labeled protein molecules of the invention; one or more ring structures can be present.

The linker reactive group Z includes any nucleophilic or electrophilic group. In a preferred embodiment Z is capable of forming a covalent bond with a reactive side chain of an antibody. In some embodiments, Z includes one or more C═O groups arranged to form a diketone, an acyl beta-lactam, an active ester, haloketone, a cyclohexyl diketone group, an aldehyde or maleimide. Other groups may include lactone, anhydride, α-haloacetamide, an amine, a hydroxylamine, a hydrazide, or an epoxide. Exemplary linker electrophilic reactive groups that can covalently bond to a reactive nucleophilic group (e.g. lysine or cysteine side chain) of a protein (e.g., an Fc portion of an antibody molecule) include acyl β-lactam, simple diketone, succinimide active ester, maleimide, haloacetamide with linker, haloketone, cyclohexyl diketone, aldehyde, amidine, guanidine, imine, eneamine, phosphate, phosphonate, epoxide, aziridine, thioepoxide, a masked or protected diketone (a ketal for example), lactam, sulfonate, and the like masked C═O groups such as imine, ketal, acetal and any other known electrophilic group. A preferred linker reactive group includes one or more C═O, groups arranged to form a acyl β-lactam, simple diketone, succinimide active ester, maleimide, haloacetamide with linker, haloketone, cyclohexyl diketone, or aldehyde. As recited above, in this embodiment the group Z is a hydroxylamine group; other alternatives are described later.

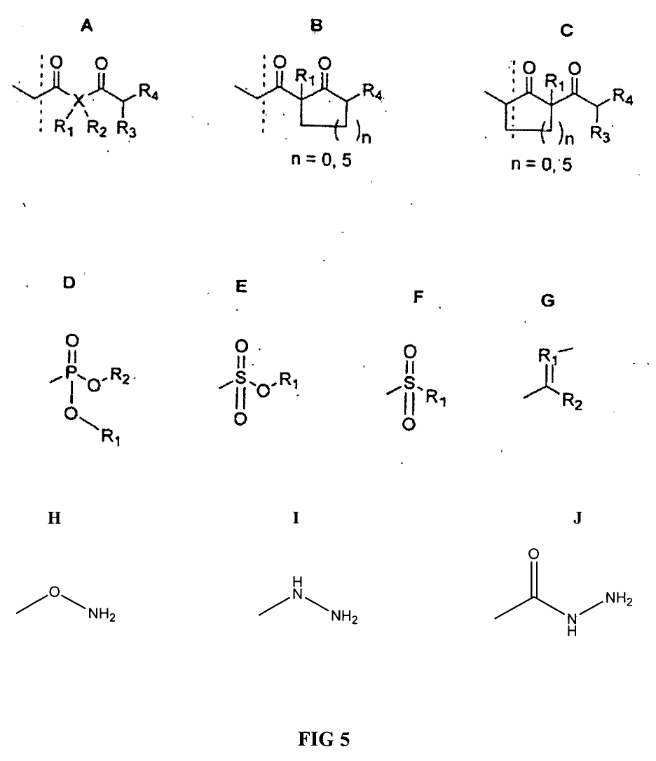

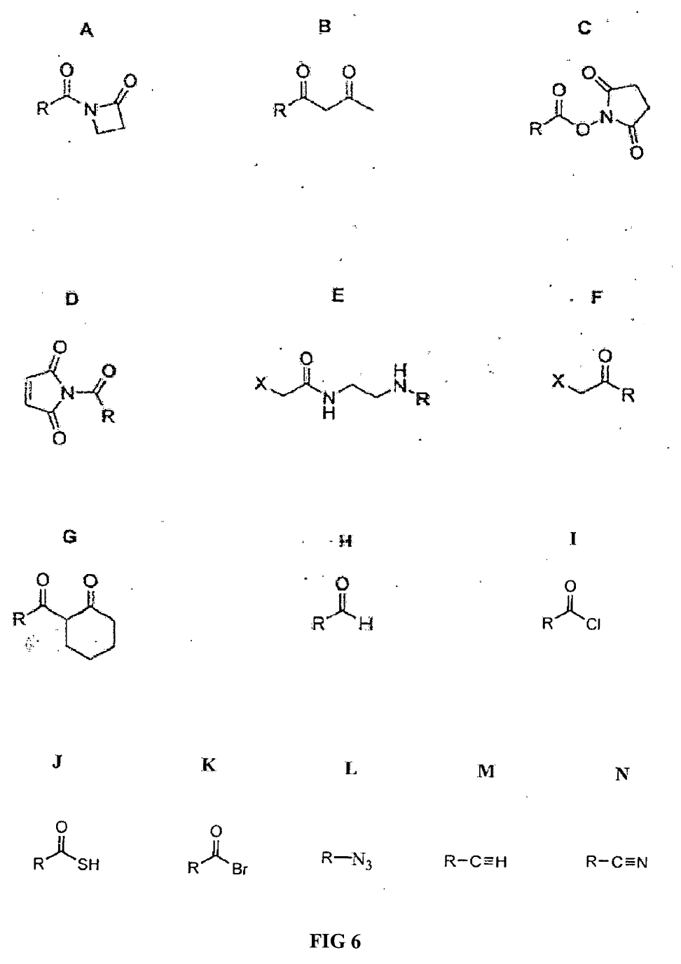

Z may be a group that forms a reversible or irreversible covalent bond. In some embodiments, reversible covalent bonds may be formed using diketone Z groups such as those shown in FIG. 5. Thus, structures A—C may form reversible covalent bonds with reactive nucleophilic groups (e.g. lysine or cysteine side chain or hydroxylamine introduced by incorporation of an unnatural amino acid) in a protein (e.g. the Fc portion of an antibody). R₁ and R₂ and R₃ in structures A—C of FIG. 5 represent substituents which can be C, H, N, O, P, S, Si, halogen (F, Cl, Br, I) or a salt thereof. These substituents also may include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, sulfoalkynyl phosphoalkyl, phosphoalkenyl, or phosphoalkynyl group. R₂ and R₃ also could form a ring structure as exemplified in structures B and C. X in FIG. 5 could be a heteroatom. Other Z groups that form reversible covalent bonds include the amidine, imine, and other reactive groups encompassed by structure G of FIG. 5, as well as the —O—NH₂ group (H), the —NH—NH₂ group (I), and the CO—NH—HN₂ group (J) of FIG. 5. FIG. 6 includes the structures of other preferred linker reactive groups that form reversible covalent bonds, e.g. structures B, G, H, I, J, K, L, and M, and, where X is not a leaving group, E and F.

Z reactive groups that form an irreversible covalent bond with a protein (e.g., the Fc portion of an antibody) include structures D—G in FIG. 5 (e.g., when G is an imidate) and structures A, C and D of FIG. 6. When X is a leaving group, structures E and F of FIG. 6 may also form irreversible covalent bonds. Such structures are useful for irreversibly attaching a targeting module-linker to a reactive nucleophilic group (e.g. lysine or cysteine side chain) in a protein (e.g. the Fc portion of an antibody).

It should be understood that the above described reversible and nonreversible covalent linking chemistry can also be applied to link a targeting module to a protein in the absence of a linker or to link a targeting module to a linker (e.g. to the connecting chain of the linker). For example, a targeting module can be linked to a linker to form a labeling agent by placing a suitable reactive group Z type element such as an appropriate nucleophilic or electrophilic group on either the linker or the targeting module and a suitable reactive moiety such as an amino or sulfhydryl group on the other of the two.

Although it is generally preferred for the protein to be coupled to a targeting module through a linker, with the targeting module plus the linker being described herein as the targeting molecule, in some applications it is possible for the protein to be coupled directly to the targeting module.

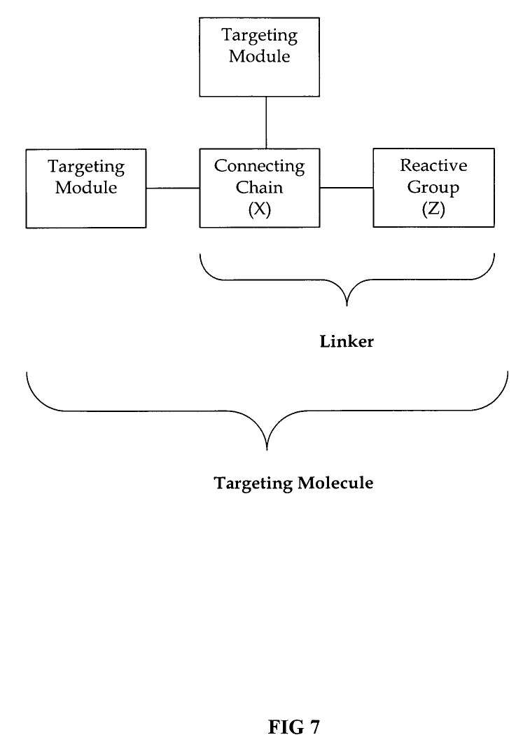

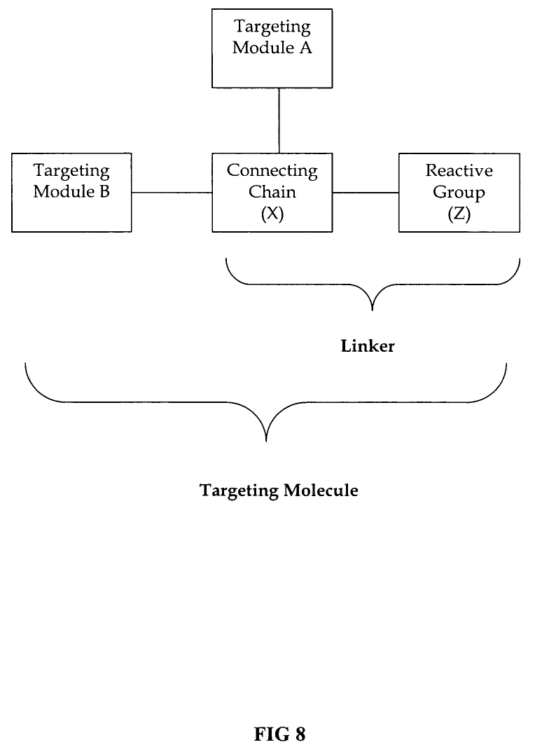

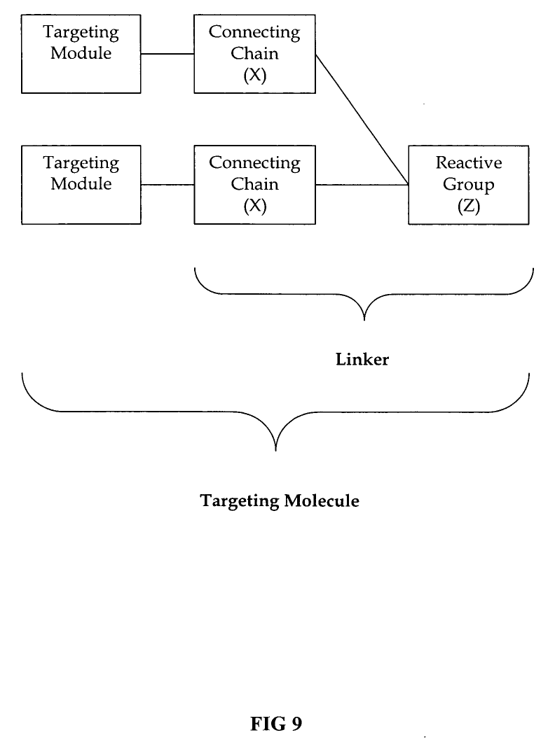

Targeting module-linker compounds of the invention include those in which two targeting modules may be attached to the X portion of the linker. The two targeting modules may be identical as shown in FIG. 7 or different as shown in FIG. 8. In FIG. 8, the two targeting modules are designated “Targeting module A” and “Targeting module B.” In addition, targeting module-linker compounds of the invention include those in which a targeting module is attached to a first X portion of the linker and a second targeting module, of the same or different structure, is attached to a second X portion of the linker. As shown in FIG. 9, the two targeting module-connecting chain structures are present in a single labeled protein molecule.

An alternative linker for use with targeting modules of the invention and for preparing targeting module-linker compounds includes a 1,3-diketone reactive group as Z. Another alternative linker is one where the connecting chain X includes a repeating ether unit of between 2-100 units. Such a linker attached to the core of a thrombospondin targeting module, or other targeting modules, such as those described above, can have the structure (I as shown below where n is from 1-100 or more and preferably is 1, 2, 3, 4 or 5, and more preferably is 3, 4 or 5. In some embodiments, the linker is a repeating polymer such as polyethylene glycol.

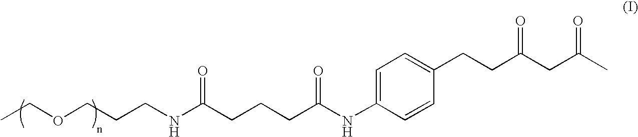

The linker reactive group or similar such reactive group that may be inherent in the targeting module is chosen for use with a particular protein. For example, a chemical moiety for modification by a hydroxylamine-bearing protein may be a ketone, aldehyde, diketone, β-lactam, active ester haloketone, lactone, anhydride, maleimide, α-haloacetamide, cyclohexyl diketone, epoxide, aidehyde, amidine, guanidine, imine, eneamine, phosphate, phosphonate, epoxide, aziridine, thioepoxide, masked or protected diketone (ketal for example), lactam, haloketone, aldehyde, and the like.

A linker reactive group chemical moiety suitable for covalent modification by a reactive sulfhydryl group in an antibody may be a disulfide, aryl halide, maleimide, alpha-haloacetamide, isocyanate, epoxide, thioester, active ester, amidine, guanidine, imine, eneamine, phosphate, phosphonate, epoxide, aziridine, thioepoxide, masked or protected diketone (ketal for example), lactam, haloketone, aldehyde, and the like.

One of skill in the art will readily appreciate that reactive amino acid side chains in proteins may possess an electrophilic group that reacts with a nucleophilic group on the targeting module or its linker, whereas in other embodiments a reactive nucleophilic group in an amino acid side chain of a protein (e.g., an Fc portion of an antibody molecule) or protein fragment reacts with an electrophilic group in a targeting module or linker. Thus, protein or protein fragment side chains may be substituted with an electrophile (e.g., FIGS. 3 and 4) and this group may be used to react with a nucleophile on the targeting module or its linker (e.g., —ONH₂). In this embodiment, the antibody and targeting module each have a partial linker with appropriate reactive moieties at each end so that the two ends of the partial linker can form the full linker, thus creating the complete labeled protein.

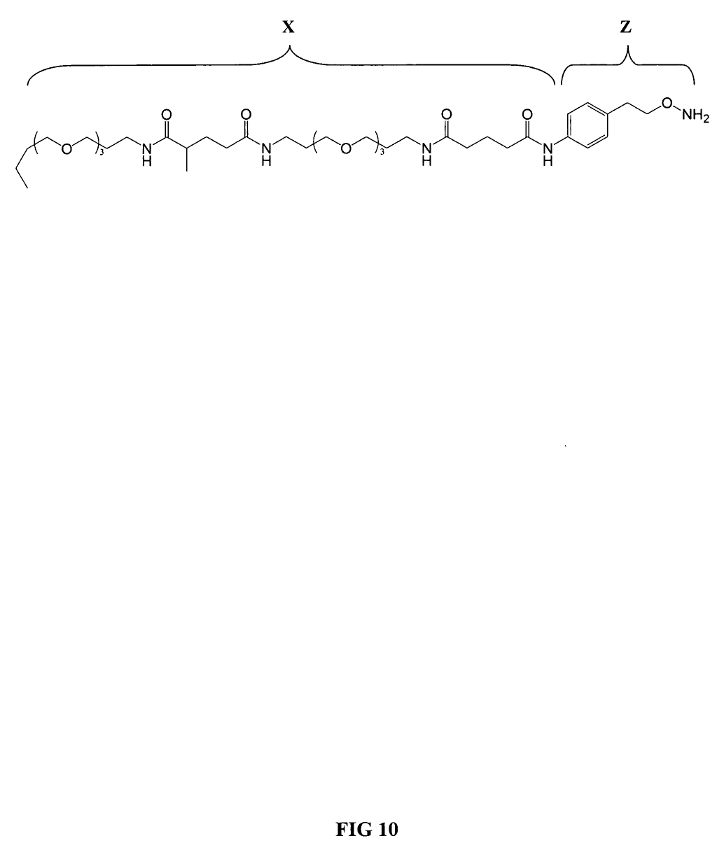

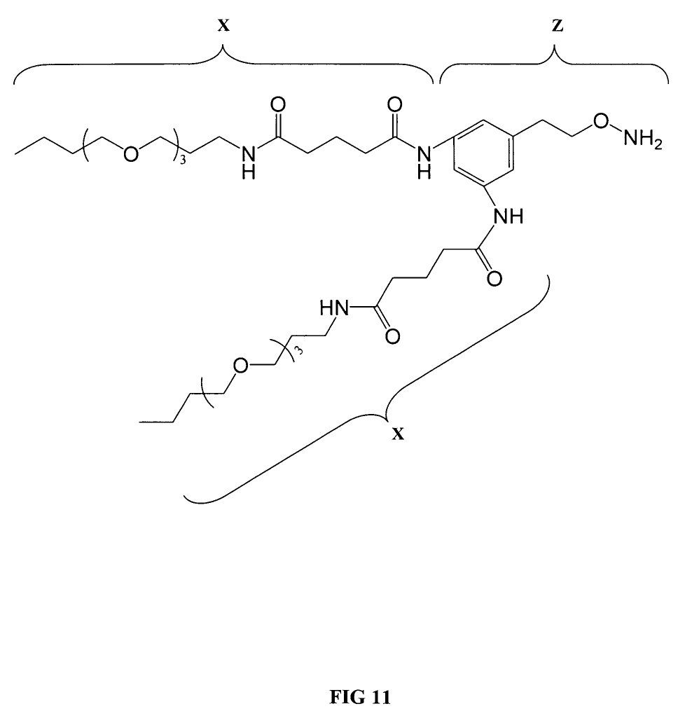

One of skill in the art also will readily appreciate that two or more targeting modules may be linked to a single protein site (e.g., an Fc portion of an antibody molecule). The two targeting modules may be the same or may be different in their structure or the signal they generate directly or indirectly. In one embodiment, each targeting module may be linked to a separate reactive side chain of an amino acid in the protein, such as the Fc portion of an antibody. In a preferred embodiment, the two targeting modules are attached to a branched or linear linker which then links both targeting modules to the same reactive amino acid side chain in the protein. Each branch of a branched linker may in some embodiments comprise a linear stretch of between 5-100 atoms. By way of example, the structures disclosed in FIGS. 10 and 11 show embodiments of branched linkers with two targeting modules linked to a different branch of the linker, which has a 1,3-diketone as the reactive group. As shown in these embodiments, the branch point may be in the connecting chain.

Although, typically, the linker is stable and is resistant to hydrolysis or other spontaneous or enzyme-catalyzed cleavage, in some alternatives, the linker moiety can be labile. The labile linkage may be between the functional component and the linker, between the targeting component and the linker, or within the linker, or combinations thereof. For example, the linker may be labile when subjected to a certain pH. The linker may also be a substrate for a particular enzyme, such as an enzyme present in body fluids. Thus, the particular design of the labile linker may be used to direct the release of the protein molecule after it has reached its intended target. A labile linker may be a reversibly covalent bond. Such linker may be an acid-labile linker such as a cis-aconitic acid linker that takes advantage of the acidic environment of different intracellular compartments such as the endosomes encountered during receptor mediated endocytosis and the lysosomes. See Shen et al., Biochem. Biophys. Res. Commun. (1981) 102:1048-1054; Yang et al., J. Natl. Canc. Inst. (1988) 80: 1154-1159. In other embodiments, a peptide spacer arm is employed as the linker so that the linker can be cleaved by the action of a peptidase such as a lysosomal peptidase. See e.g., Trouet et al., Proc. NatI. Acad. Sci. (1982) 79: 626-629.

Labile linkers include reversible covalent bonds, pH sensitive linkages (acid or base sensitive), enzyme sensitive linkages, degradation sensitive linkers, photosensitive linkers, and the like, and combinations thereof. These features are also characteristic of a prodrug which can be considered as a type of labile linker. A variety of labile linkers have been previously designed. For example, prodrugs can be formed using compounds having carboxylic acid that slowly degrade by hydrolysis as described in U.S. Pat. No. 5,498,729.

In this regard, the targeting molecule can be a “prodrug,” meaning that the targeting molecule is essentially inactive as delivered, but becomes active upon some modification. The targeting molecule can be delivered at the surface of a cell or intracellularly using the specificity of the protein molecule where it can then be activated.

Photodynamic treatment may be used to activate a prodrug by cleaving a photosensitive linker or by activating a photoresponsive enzyme (acyl enzyme hydrolysis) as described previously (see U.S. Pat. No. 5,114,851 and 5,218,137). Photodynamic treatment also may be used to rapidly inactivate a drug in sites where the drug activity is not desired (e.g. in non-target tissues). Various means of covalently modifying a drug to form a prodrug are well known in the art.

The target molecule can, in some embodiments, be a biomolecule such as a protein, carbohydrate, lipid or nucleic acid. The target molecule can be associated with a cell (“cell surface expressed”), or other particle (“particle surface expressed”) such as a virus, or may be extracellular. If associated with a cell or particle, the target molecule is preferably expressed on the surface of the cell or particle, such as a receptor, in a manner that allows the targeting molecule to make contact with the surface receptor from the fluid phase of the body.

In some preferred embodiments, the targeting molecule is predominantly or exclusively associated with a pathological condition or diseased cell, tissue or fluid. Thus, the targeting molecule can be used to deliver the labeled protein molecule to a diseased tissue by targeting the cell, an extracellular matrix biomolecule or a fluid biomolecule. Exemplary target molecules include thrombospondin receptors, such as CD36.

In synthesizing labeled proteins where a linker is present between the protein and the targeting molecule, linkage may be accomplished by several approaches. In one approach where the polymer is a protein, a targeting module-linker compound is synthesized with a linker that includes one or more reactive groups designed for covalent reaction with a side chain of an amino acid of the protein. The targeting module-linker compound and the protein are combined under conditions where the linker reactive group forms a covalent bond with the amino acid side chain.

In another approach, linking can be achieved by synthesizing a protein-linker compound comprising a protein and a linker wherein the linker includes one or more reactive groups designed for covalent reaction with an appropriate chemical moiety of a targeting module. The targeting module may need to be modified to provide the appropriate moiety for reaction with the linker reactive group. The protein-linker and targeting module are combined under conditions where the linker reactive group covalently links to the targeting module.

A further approach for forming a labeled protein according to the present invention uses a dual linker design. In this approach, a targeting module-linker compound is synthesized which comprises a targeting module and a linker with a reactive group. A protein-linker compound is also synthesized which comprises a protein and a second linker segment with a chemical group susceptible to reactivity with the reactive group of the targeting module-linker of the first step. These two linker containing compounds are then combined under conditions whereby the linkers covalently link, forming the labeled protein with a dual linker.

“Susceptible” as used herein with reference to a chemical moiety indicates that the chemical moiety will covalently bond with a compatible reactive group. Thus, an electrophilic group is susceptible to covalent bonding with a nucleophilic group and vice versa.

As discussed, the linker may be first conjugated to the targeting module and then the targeting module-linker conjugated to the protein. Alternatively, the linker may be conjugated first to the protein and the protein-linker conjugated to the targeting module. Numerous means well known in the art can be used to attach a linker to the targeting module or to the protein.

In the case of a protein molecule including the Fc portion of an antibody, the targeting module can be prepared by several approaches. In one approach, a targeting module-linker compound is synthesized with a linker that includes one or more reactive groups designed for covalent reaction with a side chain of an amino acid in the Fc portion of an antibody molecule; in some examples of this approach, the amino acid can be the amino-terminal amino acid or the carboxyl-terminal amino acid. The targeting module-linker compound and Fc portion of the antibody are combined under conditions where the linker reactive group forms a covalent bond with the amino acid side chain.

In another approach, linking can be achieved by synthesizing an Fc-linker compound comprising an Fc portion of an antibody and a linker wherein the linker includes one or more reactive groups designed for covalent reaction with an appropriate chemical moiety of the targeting module. The targeting module may need to be modified to provide the appropriate moiety for reaction with the linker reactive group. The antibody-linker and targeting module are combined under conditions where the linker reactive group covalently links to the targeting and/or biological agent.

In yet another approach, dual linkers are used as described above, one linker in a protein-linker compound and the other linker in a targeting module-linker compound, and the linkers are terminated with reactive groups that will react with each other.

Exemplary functional groups that can be involved in the linkage include, for example, esters, amides, ethers, phosphates, amino groups, keto groups, amidines, guanidines, imines, eneamines, phosphates, phosphonates, epoxides, aziridines, thioepoxides, masked or protected diketones (ketals for example), lactams, haloketones, aldehydes, thiocarbamates, thioamides, thioesters, sulfides, disulfides, phosphoramide, sulfonamides, ureas, thioureas, carbamates, carbonates, hydroxamides, and the like.

The linker includes any atom from the group C, H, N, O, P, S, Si, halogen (F, Cl, Br, I) or a salt thereof. The linker also may include a group such as an alkyl, alkenyl, alkynyl, oxoalkyl, oxoalkenyl, oxoalkynyl, aminoalkyl, aminoalkenyl, aminoalkynyl, sulfoalkyl, sulfoalkenyl, sulfoalkynyl group, phosphoalkyl, phosphoalkenyl, or phosphoalkynyl group. The linker also may include one or more ring structures. As used herein a “ring structure” includes saturated, unsaturated, and aromatic carbocyclic rings and saturated, unsaturated, and aromatic heterocyclic rings. The ring structures may be mono-, bi-, or polycyclic, and include fused or unfused rings. Further, the ring structures are optionally substituted with functional groups well known in the art including, but not limited to halogen, oxo, —OH, —CHO, —COOH, —NO₂ , —CN, —NH₂, —C(O)NH₂, C₁₋₆alkyl, C₂₋₆alkenyl, C₂₋₆alkynyl, C₁₋₆ oxoalkyl, oxoalkenyl, oxoalkynyl, aminoalkyl, aminoalkenyl, aminoalkynyl, sulfoalkyl, sulfoalkenyl, or sulfoalkynyl, phosphoalkyl, phosphoalkenyl, or phosphoalkynyl group. Combinations of the above groups and rings may also be present in the linkers of the labeled proteins of the invention.

In another alternative, the linker can include biotin or a molecule incorporating biotin with a spacer, such as biotin-LC. The use of a biotin-avidin interaction to form a spacer is well known in the art and is described, for example, in G. T. Hermanson, “Bioconjugate Techniques” (Academic Press, San Diego, 1995), pp. 570-592, incorporated herein by this reference. Various derivatives of biotin are available and can be incorporated into the linker. For example, Pierce (Rockford, Ill.) produces biotin hydrazide and biotin-LC-hydrazide, which can react directly with aldehydes to produce oximes to link the biotin moiety to the protein molecule. In place of avidin, streptavidin can be used.

In yet another alternative, the linker includes therein a carrier molecule of the general structure NH₂OCH₂-(Gly)ₓ-[Lys-H-Ser-)]_{y}-Gly-OH, wherein x is an integer from 2 to 4 and y is an integer from 4 to 6, which provides a hydroxylamine moiety for reaction with a N-terminal aldehyde functionality. Preferably, x is 3 and y is 5. These carriers are described in L. Vilaseca et al., “Protein Conjugates of Defined Structure: Synthesis and Use of a New Carrier Molecule,” Bioconiugate Chem. 4: 516-520 (1993), incorporated herein by this reference.

A labeled protein of the present invention can be prepared using techniques well known in the art. Typically, synthesis of the targeting module is the first step and is carried out as described herein. The targeting module is then derivatized for linkage to a connecting component (the linker) which is then combined with the protein. One of skill in the art will readily appreciate that the specific synthetic steps used depend upon the exact nature of the three components.

The present invention also includes methods of altering at least one physical or biological characteristic of a targeting module or linker. The methods include covalently linking the targeting module to a protein as described above. In some embodiments, the targeting module is linked to an Fc region of an antibody molecule directly or though a linker, the characteristics of which are described above. The method is particularly useful for linking small targeting modules of 5 Kd or less. However, the method also works for larger targeting modules. Characteristics of the targeting module can include binding affinity, susceptibility to degradation, such as by proteases, pharmacokinetics, pharmacodynamics, immunogenicity, solubility, lipophilicity, hydrophilicity, hydrophobicity, stability (more or less stable as well as planned degradation), rigidity, flexibility, modulation of antibody binding, fluorescence, chemiluminescence, bioluminescence, visible or ultraviolet absorption, and the like.

As used herein, pharmacokinetics refers to the concentration of an administered compound in the serum over time. Pharmacodynamics refers to the concentration of an administered compound in target and nontarget tissues over time and the effects on the target tissue (efficacy) and the non-target tissue (toxicity). Improvements in, for example, pharmacokinetics or pharmacodynamics can be designed for a particular targeting module such as by using labile linkages or by modifying the chemical nature of any linker (changing solubility, charge, etc.).

The biological characteristic of a labeled protein molecule of the invention may be modified to obtain improved pharmaceutical or other characteristics. This may be achieved by altering one or more chemical characteristics of the targeting module, the linker or the protein. A preferred approach is to chemically modify one or more chemical characteristics of the linker. By altering chemical characteristics of the compound including the linker, one can obtain improved features such as improvement in pharmacokinetics, pharmacodynamics, solubility, immunogenicity and the like.

In these methods, if the protein molecule includes a receptor binding domain, the labeled protein molecule can be visualized using methods such as fluorescence-activated cell sorting (FACS). The resulting labeled protein molecule or “conjugate” is expected to be stable and to circulate with a half-life substantially equivalent to the normal half-life of the Fc region.

Typically, the protein molecule, including the Fc region, is expressed in a manner such that the naturally-occurring pattern of glycosylation of the protein molecule is substantially maintained. If the naturally-occurring pattern of glycosylation is substantially maintained, Fc-mediated effector functions, such as complement activation and antibody-dependent cellular cytotoxicity (ADCC) can be activated.

In order to substantially retain the naturally-occurring pattern of glycosylation, it is preferred to express the protein molecule in a eukaryotic host that can carry out glycosylation. These hosts include, but are not limited to, Chinese hamster ovary (CHO) cells and 293 cells. In some applications, in which effector functions such as ADCC and complement fixation are not required, it is preferred to express the protein molecule in a prokaryotic host such as Escherichia coli or Salmonella typhimurium, or, alternatively mutate the Fc so as to remove the glycosylation site.

In another embodiment of the invention, the protein molecule to be labeled is translated such that it includes therein an aldehyde or keto functionality as a side chain of an amino acid within the protein molecule, without the requirement of oxidation. This protein molecule is generated by translational incorporation of an unnatural amino acid bearing the aldehyde or keto functionality. These amino acids include, but are not limited to, β-oxo-α-aminobutyric acid and (2-ketobutyl)-tyrosine. This approach has been described in V. W. Cornish et al., “Site-Specific Protein Modification Using a Ketone Handle,” J. Am. Chem. Soc. 118: 8150-8151 (1996), incorporated herein by this reference.

Therefore, in this embodiment, the method for labeling the protein molecule comprises the steps of:
(1) providing a protein molecule that includes therein the Fc portion of an antibody molecule, the molecule having at least one amino acid including therein a side chain with aldehyde or keto functionality; and
(2) reacting the aldehyde or keto functionality of the protein molecule with a targeting molecule including therein a group reactive with an aldehyde or keto functionality to produce a labeled protein molecule such that the targeting molecule solely directs the targeting of the labeled protein molecule to a target that is a soluble molecule or a cell-surface molecule.

As described above, the targeting molecule typically includes a hydroxylamine moiety or a hydrazide moiety.

In this embodiment, the protein molecule is as described above; the targeting molecule and any linker used are also as described above. The full range of targeting molecules, including those targeting integrins, can be used in these reactions.

In another embodiment, the protein molecule can be linked to the targeting molecule using copper(I)-catalyzed azide-alkyne [3+2] cycloaddition, as described in A. E. Spears et al., “Activity-Based Protein Profiling in Vivo Using a Copper(I)-Catalyzed Azide-Alkyne [3+2] Cycloaddition,” JACS Commun. 125: 4686-4687 (2003), incorporated herein by this reference. This coupling technique is referred to herein as “click chemistry.”

This reaction can be used to couple a wide range of targeting molecules and protein molecules. For example, the diketone targeting molecules described in U.S. Patent Application Publication No. 2003/0129188 by Barbas et al., in U.S. Patent Application Publication No. 2003/0190676 by Barbas et al., and in U.S. Patent Application Publication No. 2003/0175921 by Barbas et al., all incorporated herein by this reference, can be used in this reaction if the molecules are modified to terminate in an azide or alkyne moiety instead of a diketone moiety.

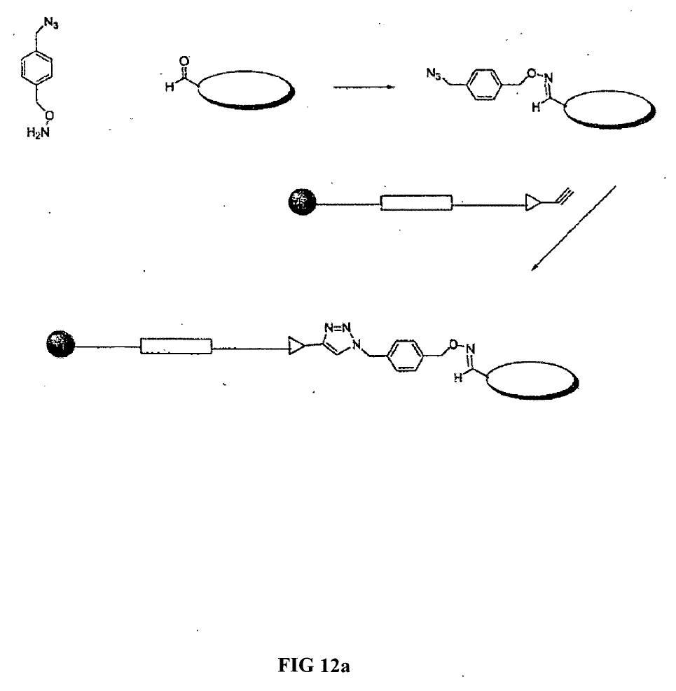

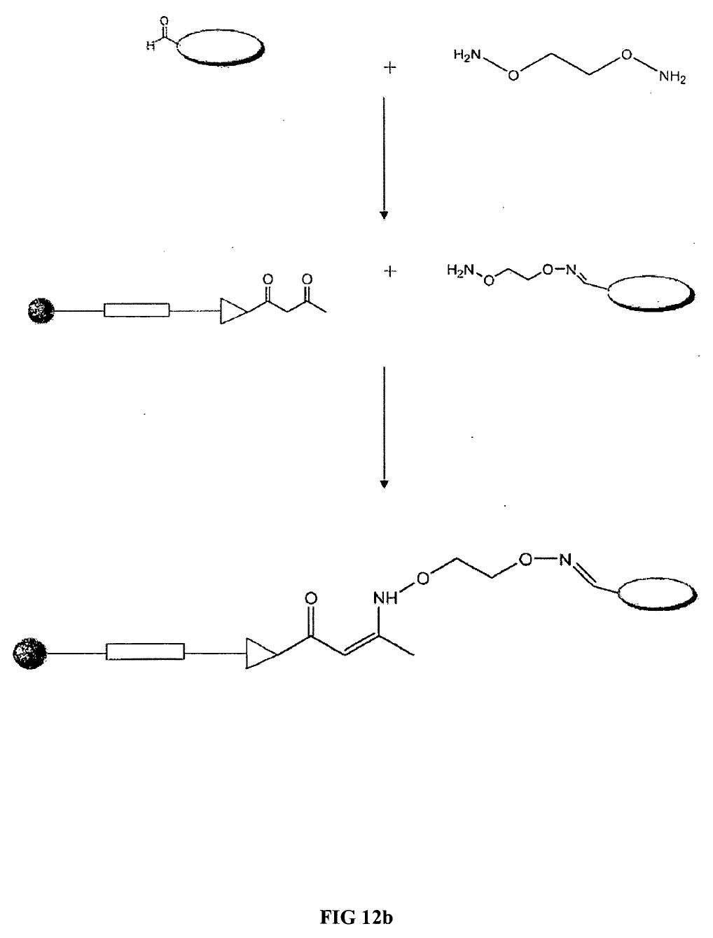

This reaction is depicted schematically in FIG. 12a. FIG. 12a is a depiction of a two-step construction of a labeled protein molecule including an Fc region. First, the aldehyde-containing Fc protein is reacted with a hydroxylamine bearing an azide functionality to provide an azide-Fc. The azide-Fc can then be reacted with a wide variety of targeting molecules including a targeting module, a linker, and a reactive group wherein the reactive group includes an alkyne. A copper (I)-catalyzed azide-alkyne [3+2] cycloaddition reaction then produces the labeled protein molecule including the Fc region. Notice that the azide-Fc could also be prepared by translational incorporation of a non-naturally-occurring amino acid bearing a reactive azide group.

In general, this embodiment comprises the steps of:
(1) providing a protein molecule that includes therein the Fc portion of an antibody molecule, the protein molecule having a reactive amino acid residue selected from the group consisting of an azide-substituted amino acid residue and an alkyne-substituted amino acid residue;
(2) providing a targeting molecule, the targeting molecule having a reactive amino acid residue selected from the group consisting of an azide-substituted amino acid residue and an alkyne-substituted amino acid residue such that the protein molecule and the targeting molecule, taken together, have an azide-substituted amino acid residue and an alkyne-substituted amino acid residue; and
(3) reacting the protein molecule with the targeting molecule by azide-alkyne [3+2] cycloaddition to produce a labeled protein molecule such that the targeting molecule solely directs the targeting of the labeled protein molecule to a target that is a soluble molecule or a cell-surface molecule.

In this approach, typically, the targeting molecule is a protein attached to a linker, although it could be a non-protein moiety substituted with the required reactive amino acid. The reactive amino acids that can be used include, but are not limited to, α-amino-γ-azidobutyric acid and α-amino-γ-methynylbutyric acid. Other pairs of reactive amino acids, one with an azide substituent and the other with an alkyne substituent, can be used. Alternatively, the protein molecule could be coupled directly to a targeting module, without a linker. In still another alternative, as disclosed in FIG. 12a, an amino-terminal amino acid that contains an aldehyde group, or is oxidized to contain an aldehyde group, is first reacted with a hydroxylamine including an azide functionality to generate the azide-containing group for the azide-alkyne cycloaddition. The amino-terminal acid that contains the aldehyde group can be a non-naturally-occurring amino acid as discussed above. Alternatively, it can be produced by oxidation of an amino-terminal serine residue, as discussed above.

In another alternative approach, an amino acid residue that contains or is oxidized to contain an aldehyde group is reacted with one of the amino groups of a substituted bifunctional hydroxylamine linker to produce a C═N double bond to the linker. The free, second, amino group of the linker is then reacted with a substituted diketone. This approach is shown in FIG. 12b, with the other components of the labeled protein molecule depicted in the same way as in FIG. 12a.

In general, this method comprises:
(1) providing a protein molecule that includes therein the Fc portion of an antibody molecule, the protein molecule having a reactive aidehyde residue;
(2) reacting the aldehyde residue with a bifunctional hydroxylamine linker having two H₂N—O— moieties, the aldehyde residue forming a C═N bond with one of the moieties; and
(3) reacting the other H₂N—O— moiety of the bifunctional hydroxylamine linker with a targeting molecule having a diketone moiety to produce a labeled protein molecule such that the targeting molecule solely directs the targeting of the labeled protein molecule to a target that is a soluble molecule or a cell-surface molecule,

Yet another alternative approach is described in J. H. van Maarseveen & J. W. Back, “Re-Engineering the Genetic Code: Combining Molecular Biology and Organic Chemistry,” Angew. Chem. Int. Ed. 42: 5926-5928 (2003), incorporated herein by this reference. This approach uses Staudinger ligation to couple an azido group in the protein molecule with a targeting molecule that is covalently linked to an ortho-disubstituted aromatic moiety, one substituent being carbomethoxy and the other substitutent being diphenylphosphino. The resulting conjugate (labeled protein molecule) then has one substituent of the aromatic moiety being diphenylphosphinyl and the other substituent being a carboxamide moiety, with the nitrogen of the carboxamide moiety being linked to the protein to be labeled. The Staudinger ligation reaction is described in K. L. Kiick et al., “Incorporation of Azides Into Recombinant Proteins for Chemoselective Modification by the Staudinger Ligation,” Proc. Natl. Acad. Sci. USA 99: 19-₂₄ (2002), incorporated herein by this reference.

Therefore, another method according to the present invention is a method for labeling a protein molecule that includes therein the Fc portion of an antibody molecule comprising the steps of:
(1) providing a protein molecule that includes therein the Fc portion of an antibody molecule, the molecule having at least one amino acid including therein a side chain with azido functionality; and
(2) in a Staudinger ligation reaction, reacting the azido functionality of the protein molecule with a targeting molecule that is covalently linked to an ortho-disubstituted aromatic moiety, one substituent being carbomethoxy and the other substitutent being diphenylphosphino, to produce a labeled protein molecule, such that the labeled protein molecule has one substituent of the aromatic moiety being diphenylphosphinyl and the other substituent being a carboxamide moiety, with the nitrogen of the carboxamide moiety being linked to the protein molecule such that the targeting molecule solely directs the targeting of the labeled protein molecule to a target that is a soluble molecule or a cell-surface molecule.

Still other alternatives for coupling reactions are known and are described, for example, in L. Wang & P. G. Schultz, “Expanding the Genetic Code,” Angew. Chem. Int. Ed. 44: 34-66 (2005), incorporated herein by this reference. These involve reactions between the unnatural amino acids p-acetylphenylalanine or m-acetylphenylalanine and a hydrazide, alkoxyamine, or semicarbazide to produce hydrazone, oxime, or semicarbazone linkages that are stable.

Accordingly, another embodiment of the invention is a method for labeling the protein molecule that comprises the steps of:
(1) providing a protein molecule that includes therein the Fc portion of an antibody molecule, the molecule having an amino acid selected from the group consisting of p-acetylphenylalanine and m-acetylphenylalanine; and
(2) reacting the amino acid selected from the group consisting of P-acetylphenylalanine and m-acetylphenylalanine of the protein molecule with a targeting molecule containing a reactive moiety selected from the group consisting of a hydrazide, an alkoxyamine, and a semicarbazide to produce a labeled protein molecule such that the targeting molecule solely directs the targeting of the labeled protein molecule to a target that is a soluble molecule or a cell-surface molecule.

The protein molecules and targeting molecules are as described above. The reactive moiety (hydrazide, alkoxyamine, or semicarbazide) in the targeting molecule can either be incorporated in the targeting molecule or can be incorporated in a linker or a reactive module attached to the linker, as described above with respect to the formation of labeled protein molecules by reaction of a hydroxylamine-containing moiety with an aldehyde or keto group.

In another embodiment of the invention, the labeled protein molecule is produced by the reaction of a protein molecule that includes an amino acid residue reactive with an electrophile with a targeting molecule that includes an electrophile reactive with the amino acid residue. Therefore, in general, this method comprises the steps of:
(1) providing a protein molecule that includes therein the Fc portion of an antibody molecule, the protein molecule having a reactive amino acid residue reactive with an electrophile;
(2) providing a targeting molecule that includes an electrophile reactive with the amino acid residue; and
(3) reacting the targeting molecule with the protein molecule by reacting the reactive amino acid residue with the electrophile to produce the labeled protein molecule such that the targeting molecule solely directs the targeting of the labeled protein molecule to a target that is a soluble molecule or a cell-surface molecule.

Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference.

The reactivity of the cysteine residues can be optimized by appropriate selection of the neighboring amino acid residues. For example, a histidine residue adjacent to the cysteine residue will increase the reactivity of the cysteine residue.

Other combinations of reactive amino acids and electrophilic reagents are known in the art. For example, maleimides can react with amino groups, such as the gamino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the Famino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G.T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques” (Academic Press, San Diego, 1996), pp. 154-158, incorporated herein by this reference.

In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Accordingly, this method comprises the steps of:
(1) providing a protein molecule that includes therein the Fc portion of an antibody molecule, the protein molecule having a reactive amino acid residue including therein an electrophilic group reactive with a nucleophile;
(2) providing a targeting molecule that includes a nucleophile reactive with the amino acid residue; and
(3) reacting the targeting molecule with the protein molecule by reacting the reactive amino acid residue with the nucleophile to produce the labeled protein molecule such that the targeting molecule solely directs the targeting of the labeled protein molecule to a target that is a soluble molecule or a cell-surface molecule.

In yet another embodiment of the invention, the protein to be labeled includes therein a mutated haloalkane dehalogenase domain and the targeting molecule or targeting module includes a reactive haloalkane moiety. The action of the mutated haloalkane dehalogenase results replacement of the hydrogen of the carboxyl side chain of one of the aspartate residues in the mutated haloalkane dehalogenase domain with an alkyl moiety derived from the reactive haloalkane moiety, forming a stable ester. This is described, for example, in U.S. Patent Application Publication Serial No. 2004/0214258 by Wood et al., incorporated herein by this reference, and in “HaloTag™ Interchangeable Labeling Technology” (Promega Corp., Madison, Wis., November 2004), incorporated herein by this reference.

Accordingly, in this embodiment of the invention, the method comprises the steps of:
(1) providing a protein molecule that includes therein the Fc portion of an antibody molecule, the protein molecule having a mutated haloalkane dehalogenase domain therein, the mutated haloalkane dehalogenase domain having therein an aspartate residue, the side chain of the aspartate residue being capable of esterification; and
(2) reacting the protein molecule with a targeting molecule having a reactive haloalkane moiety to form a stable ester to produce a labeled protein molecule such that the targeting molecule solely directs the targeting of the labeled protein molecule to a target that is a soluble molecule or a cell-surface molecule.

Accordingly, therefore, protein molecules suitable for labeling in methods according to the present invention include protein molecules with Fc regions that have an amino-terminal serine, an amino-terminal cysteine, or other amino-terminal reactive amino acids as described above. Methods for generating these protein molecules are described below. The biological activity of a peptide expressed as a direct fusion with an Fc is shown in J. Oliner et al., “Suppression of Angiogenesis and Tumor Growth by Selective Inhibition of Angiopoietin-2, ”Cancer Cell 6: 507-516 (2004), incorporated herein by this reference. The biological activity of a receptor expressed as a direct fusion with an Fc is shown in J. Holash et al., “VEGF-Trap: A VEGF Blocker with Potent Antitumor Effects,” Proc. Natl. Acad. Sci. USA 99: 11393-11398 (2002), incorporated herein by this reference. These protein molecules would then be used in methods according to the present invention by reacting them with an appropriate targeting module containing a reactive group that could react with the reactive amino acid residue of the protein molecule, as described above. In another alternative, the VEGF receptor can be expressed with an amino acid residue incorporating an azide moiety and this modified VEGF receptor can then be coupled to a Fc molecule expressed with an amino acid residue incorporating an alkyne moiety by this “click chemistry” reaction.

As an alternative, the peptide, receptor, or other active peptide or protein moiety can be coupled to the Fc by click chemistry as described above to form a fusion protein. This can also be accomplished by using an aldehyde-containing amino acid, either introduced by translation or oxidation of a serine residue, and reacting the aldehyde-containing amino acid with an azide-containing hydroxylamine moiety, as described above. Other coupling methods can be used.

In yet another alternative, the Fc can have both a reactive amino terminus and a reactive carboxyl terminus, with the proviso that the reactive amino terminus does not react with the reactive carboxyl terminus. A targeting molecule or a component of a fusion protein can then be added to either end of the Fc using the oxidation approach at one end and the “click chemistry” approach at the other. For example, an Fc could be constructed with an azido amino acid at both the carboxyl and amino termini, and then an IL-2 cytokine that has an alkyne-substituted amino acid could be coupled by click chemistry. Alternatively, an scFv bearing an alkyne could be coupled on to both ends by click chemistry. Other combinations are possible. In general, these domains and protein molecules can be used in a modular approach, applying these coupling reactions, with the proviso that at least one targeting molecule is coupled.

Accordingly, this method comprises the steps of:
(1) providing a protein molecule that includes therein the Fc portion of an antibody molecule, the protein molecule having a first reactive amino acid at its amino-terminus and a second reactive amino acid at its carboxyl-terminus;
(2) reacting a first molecule selected from the group consisting of a targeting molecule and a component of a fusion protein with the first reactive amino acid to link the first molecule to the protein molecule; and
(3) reacting a second molecule selected from the group consisting of a targeting molecule and a component of a fusion protein with the second reactive amino acid to link the second molecule to the protein molecule;
   with the proviso that the first reactive amino acid does not react with the second reactive amino acid and such that the targeting molecule solely directs the targeting of the labeled protein molecule to a target that is a soluble molecule or a cell-surface molecule, with the proviso that at least one targeting molecule is coupled.

In one alternative, at least one of the first and second reactive amino acids is selected from the group consisting of an azido-substituted amino acid and an alkyne-substitute amino acid. In another alternative, at least one of the first and second reactive amino acids is selected from the group consisting of an amino-terminal serine residue and an amino acid residue with a side chain with aidehyde or keto functionality.

Typically, in this approach, only one of the first and second molecules are targeting molecules, although, in some approaches, it might be desirable to use dual targeting molecules.

II. Labeled Protein Molecules

Another aspect of the present invention is a labeled protein molecule labeled by the methods of the present invention such that the targeting molecule directs the targeting of the labeled protein molecule to a target, as described above.

The labeled protein molecule can include an Fc portion of an antibody. For example, the labeled protein molecule can include any of these arrangements of antibody domains: C_{H}3 alone; C_{H}2—C_{H}3; C_{H}1—C_{H}2—C_{H}3 paired with C_{L}; hinge-C_{H}2'C_{H}3; C_{H}1-hinge-C_{H}2—C_{H}3 paired with C_{L}; hinge-C_{H}3; C_{H}2—C_{H}3;

Alternatively, the labeled protein molecule can include an intact antibody molecule as described above, with the provisos described above on the attachment of the targeting molecule to the labeled protein molecule and such that the targeting molecule directs the targeting of the labeled protein molecule to a target.

In still another alternative, the labeled protein molecule can include another protein moiety of the immunoglobulin superfamily as described above.

The labeled protein molecule is typically linked at the N-terminus of the Fc portion to a targeting molecule (i.e., through a linker) or to a targeting module (without the linker). Suitable linkers, targeting molecules, and targeting modules are described above As described above, the linker can be a dual linker, produced by the covalent linkage of two linkers, one originally attached to the protein molecule and the other originally attached to the targeting module.

If the labeled protein molecule is covalently linked at the N-terminus of the Fc portion to a targeting module or targeting molecule, the labeled protein molecule can optionally be also linked at the C-terminus of the Fc portion to another protein, a peptide, or a domain from another protein, as described above. Various coupling reactions are possible.

In another alternative, the labeled protein molecule can include therein an unnatural amino acid bearing an aldehyde or keto functionality on a side chain, as described above.

In still another alternative, the labeled protein molecule includes azide-substituted and alkyne-substituted amino acids that are covalently coupled by azide-alkyne [3+2] cycloaddition as described above. In this alternative, the protein includes one of the azide-substituted or alkyne-substituted amino acids, and the targeting molecule or targeting module includes the other of the azide-substituted or alkyne-substituted amino acids. As described above, the azide-substituted amino acid can be produced by the reaction of an aldehyde-containing amino acid with an azide-substituted hydroxylamine.

In still another alternative, as described above, the labeled protein molecule includes an azido group in the protein molecule that is coupled to a targeting molecule or targeting module that is covalently linked to an ortho-disubstituted aromatic moiety, one substituent being diphenylphosphinyl and the other substituent being a carboxamide moiety, with the nitrogen of the carboxamide moiety being linked to the protein.

In still another alternative, the labeled protein molecule includes one of the unnatural amino acids p-acetylphenylalanine or m-acetylphenylalanine, which is then linked to the targeting molecule or targeting module by reaction with a hydrazide, alkoxyamine, or semicarbazide to produce hydrazone, oxime, or semicarbazone linkages that are stable.

In still another alternative, the labeled protein molecule includes a mutated N-terminal amino acid so that the N-terminal amino acid is reactive with an electrophile. This mutated N-terminal amino acid is typically cysteine, but can alternatively be lysine, histidine, or methionine; in some alternatives, the mutated N-terminal amino acid can be aspartate or glutamate. The N-terminal amino acid is then coupled to a targeting molecule or a targeting module by a reaction of the electrophile with the amino acid as described above.

In yet another alternative, the labeled protein molecule includes therein a mutated haloalkane dehalogenase domain and the targeting molecule or targeting module a haloalkane moiety that is coupled to the carboxyl side chain of one of the aspartate residues of the mutated haloalkane dehalogenase domain.

The labeled protein molecule can be glycosylated, as described above. Typically, the labeled protein molecule substantially retains its naturally-occurring pattern of glycosylation. As used herein, the term “substantially retains its naturally-occurring pattern of glycosylation” is defined as describing a protein molecule that retains all biological functions that are associated with its naturally-occurring pattern of glycosylation and is detected by all reagents that detect specific glycosylation patterns or specific sugar residues, including antibodies.

Labeled protein molecules as described above, and proteins that are used to generate labeled protein molecules as described above, can include or can be modified to include non-natural amino acids as described in U.S. Patent Application Publication No. 2006/0194256 to Miao et al., incorporated herein in its entirety by this reference. These non-natural amino acids are in addition to the ones described above; the labeled protein molecules and proteins that are used to generate the labeled protein molecules can contain either or both of the non-natural amino acids described above and those described in U.S. Patent Application Publication No. 2006/0194256 to Miao et al. These can include, but are not limited to, amino acids having carbonyl, dicarbonyl, acetal, hydroxylamino, or oxime side chains, or protected or masked carbonyl, dicarbonyl, hydroxylamino, or oxime side chains. These non-natural amino acids can be further linked to polyethylene glycol (PEG) chains or other water-soluble polymer chains, such as, but not limited to, polyethylene glycol propionaldehyde and derivatives thereof, monomethoxy-polyethylene glycol, polyvinyl pyrrolidone, and other polymers. These non-natural amino acids can also be variously substituted. These non-natural amino acids can be incorporated directly into a protein using strategies described in U.S. Patent Application Publication No. 2006/0194256 to Miao et al. as well as strategies described above, or can be produced by post-translational modification.

Labeled protein molecules prepared according to the methods described above can be used for both diagnostic and therapeutic purposes. In particular, they can be used in vivo for therapy and diagnostic imaging, as well as in vitro for immunostaining and immunolabeling.

In particular, one method of use of labeled protein molecules according to the present invention is a method of delivering a labeled protein molecule that effects a biological activity to cells, tissue extracellular matrix biomolecule or a biomolecule in the fluid of an individual, wherein the method comprises administering to the individual a labeled protein molecule as described above, wherein the labeled protein molecule is specific for the cells, tissue extracellular matrix biomolecule or fluid biomolecule and wherein the labeled protein molecule effects a biological activity.

In one alternative, the biological activity is one mediated by the Fc portion of an antibody molecule, such as complement activation or antibody-dependent cellular cytotoxicity. Alternatively, the biological activity can be one mediated by the targeting module, particularly if the targeting module is a protein or a nucleic acid, or has cytotoxic activity, or has drug activity, such as antineoplastic activity, antibacterial activity, antifungal activity, antiviral activity, anti-inflammatory activity, anesthetic activity, analgesic activity, hormonal activity, or other biological activity.

Another method of use of labeled proteins according to the present invention is a method of treating or preventing a disease or condition in an individual wherein the disease or condition involves cells, tissue or fluid that expresses a target molecule, the method comprising administering to the individual a therapeutically effective amount of a labeled protein molecule as described above, wherein the labeled protein molecule is specific for the target molecule and wherein the labeled protein molecule effects a biological activity effective against the disease or condition.

Yet another method of use of labeled proteins according to the present invention is a method of imaging cells or tissue in an individual wherein the cells or tissue being imaged expresses a molecule bound by the targeting module of a labeled protein according to the present invention, the method comprising the steps of:
(1) administering to the individual a labeled protein according to the present invention as described above; and
(2) detecting the labeled protein bound to the molecule bound to the targeting module.

A labeled protein of the present invention can be administered as a pharmaceutical or medicament that includes a labeled protein of the invention formulated with a pharmaceutically acceptable carrier. Therefore, another aspect of the invention is a pharmaceutical composition comprising: (1) a labeled protein according to the present invention in an effective amount; and (2) a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, sodium citrate, and the like.

Alternatively, compounds may be encapsulated, tableted or prepared in an emulsion or syrup for oral administration. Pharmaceutically acceptable solid or liquid carriers may be added to enhance or stabilize the composition, or to facilitate preparation of the composition, Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation may be in the form of a syrup, elixir, emulsion, or an aqueous or non-aqueous suspension. For rectal administration, the invention compounds may be combined with excipients such as cocoa butter, glycerin, gelatin or polyethylene glycols and molded into a suppository.

Compounds of the invention may be formulated to include other medically useful drugs or biological agents. The compounds also may be administered in conjunction with the administration of other drugs or biological agents useful for treatment of the disease or condition that labeled proteins according to the present invention are administered to treat.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. Dosage levels typically fall in the range of about 0.001 up to 100 mg/kg/day; with levels in the range of about 0.05 up to 10 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The composition may be administered as a bolus, or slowly infused.

The administration of a labeled protein to an immunocompetent individual may result in the production of antibodies against the labeled protein, depending on the origin of the components of the labeled protein. Such antibodies may be directed to the Fc portion of the antibody itself or to other regions of the labeled protein, such as any linker used in the production of the labeled protein. Reducing the immunogenicity of the antibody-targeting agent conjugate can be addressed by methods well known in the art such as by attaching long chain polyethylene glycol (PEG)-based spacers, and the like, to the antibody-targeting agent. Long chain PEG and other polymers are known for their ability to mask foreign epitopes, resulting in the reduced immunogenicity of therapeutic proteins that display foreign epitopes (Katre et al., 1990, J. Immunol. 144, 209-213; Francis et al., 1998, Int. J. Hematol. 68, 1-18). As noted, PEG can be a linker as well, thus providing both linker function and reduced immunogenicity in a targeting compound of the invention. Alternatively, or in addition, the individual administered the labeled protein may be administered an immunosuppressent such as cyclosporin A, anti-CD3 antibody, and the like, as appropriate to the medical status of the patient and the condition being treated.

III. Mutated Proteins Or Fusion Proteins, Nucleic Acid Sequences Encoding Them, And Methods For Their Expression And Selection

Another aspect of the present invention is mutated proteins or fusion proteins for incorporation into labeled proteins as described above, nucleic acid sequences encoding the mutated proteins, and methods for their expression and selection.

Mutated proteins can include proteins with naturally-occurring amino acids that are not found in the corresponding positions of the naturally-occurring Fc proteins or portions thereof, such as N-terminal serine, N-terminal cysteine, N-terminal lysine, N-terminal histidine, N-terminal methionine, N-terminal aspartate, and N-terminal glutamate, as described above.

Methods for the generation and selection of these proteins are well known in the art and need not be set forth in detail here. One general method involves phage display using randomized residues, as described, for example, in U.S. Pat. No. 6,096,551 to Barbas et al., incorporated herein by this reference. Generally libraries will be subjected to selection using the pComb3 phage display system with the compounds described above supported on the surface of microtiter plates. In selections using phage, more than one library and multiple compounds for the selection can be tested at the same time. To eliminate noncovalent binding, during phage selection, acidic washing conditions that denature proteins and peptides are typically used, so noncovalently bound phage will be washed away and only protein or peptide phage bound covalently to the compound will remain on the surface (F. Tanaka et al., “Development of Small Designer Aldolase Enzymes: Catalytic Activity, Folding, and Substrate Specificity,” Biochemistry 44: 7583-7592 (2005); F. Tanaka & C. F. Barbas III, “Phage Display of Peptides Possessing Aldolase Activity,” Chem. Commun. 2001: 769-770.). Bound phage can be recovered from the plate by the treatment with trypsin and the recovered phage can be amplified. When phage bind through a covalent bond, acidic washing does not affect their binding and covalently bound protein- and peptide-phage can be recovered by treatment with trypsin. For serine, this residue at the N-terminus is converted to an aldehyde by oxidation for screening. For N-terminal cysteine, reaction with compounds like maleimides or pyridyl disulfides provides for their selection from libraries. In this context, and only in this context, the selection process can be improved by using a recognition group coupled to the linker and the targeting module. The structure and use of such recognition groups has been previously described, for example, in PCT Patent Application Publication No. WO/03/59251 by Barbas et al., incorporated herein by this reference. Other selection methods involving phage display are also known in the art and are described, for example, in C. F. Barbas Ill et al., “Phage Display: A Laboratory Manual” (Cold Spring Harbor Laboratory Press, Cold Spring Harbor, N.Y., 2001), incorporated herein by this reference. Typically, such selection methods involve successive rounds of selection referred to as “panning.” Selection can be performed by techniques such as ELISA by binding the appropriate target to a solid support as is generally used in the art; for example, if the target is an integrin, the integrin can be bound to the solid support. Phage display libraries can be generated, for example, by the generation of small random libraries representing the addition of amino acids at the C-terminal or the N-terminal of the protein to be generated and selected for. These can include non-naturally-occurring amino acids. The resulting reactive amino acids that are incorporated within members of the phage display libraries can be readily identified and reacted with appropriate reagents specific for the particular side chain of that amino acid, as described above.

Mutated proteins can also include proteins with non-naturally-occurring amino acids, such as azide-substituted or alkyne-substituted amino acids, p-acetylphenylalanine or m-acetylphenylalanine, β-oxo-α-aminobutyric acid, or (2-ketobutyl)-tyrosine, as described above, or other non-naturally occurring amino acids such as those described in U.S. Patent Application Publication No. 2006/0194256 to Miao et al. In these proteins, the non-naturally-occurring amino acid is located such that the mutated protein can be covalently linked to a targeting molecule.

Methods for incorporation of non-naturally-occurring amino acids into proteins are described, for example, in L. Wang & P. G. Schultz, “Expanding the Genetic Code,” Angew. Chem. Int. Ed. 44: 34-66 (2005), incorporated herein by this reference. These typically involve the preparation of altered suppressor tRNAs that recognize what are normally stop codons. Other methods for incorporation of non-naturally-occurring amino acids are known in the art, such as methods described in U.S. Patent Application Publication No. 2006/0194256 to Miao et al.

Alternatively, the proteins for incorporation into the labeled proteins can be fusion proteins, as described above. Fusion protein technology is well known in the art and is described, for example, in U.S. Patent Application Publication No. 2005/0148075 to Barbas, incorporated herein by this reference. The fusion protein can include, for example, a mutated haloalkane dehalogenase domain, as described above, a purification tag, another antibody or portion thereof, an enzyme, receptor, or other protein or protein domain of defined function.

Also within the scope of the present invention are mutated proteins that differ from the mutated proteins disclosed above by no more than two additional conservative amino acid substitutions that substantially retain all activities of those mutated proteins before the introduction of conservative amino acid substitutions, including the receptor-binding capabilities of any Fc portions and the ability to be linked to a targeting molecule. The additional conservative amino acid substitutions are exclusive of the alteration of the amino acid at the amino-terminus or the substitution of a non-naturally-occurring amino acid. In the case of substantially retaining the receptor-binding capabilities of any Fc portions, this is defined so that the variant has a binding affinity for the desired receptor of at least 80% as great as the polypeptide before the substitutions are made. In terms of dissociation constants, this is equivalent to a dissociation constant no greater than 125% of that of the polypeptide before the substitutions are made. In this context, the term “conservative amino acid substitution” is defined as one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or IIe; Phe/Met or Leu or Tyr; Ser/Thr; Thr/Ser; Trp/Tyr; Tyr/Trp or Phe; Vat/Ile or Leu. Preferably, the polypeptide differs from the polypeptides described above by no more than one conservative amino acid substitution.

Another aspect of the present invention is nucleic acid sequences encoding the mutated proteins and fusion proteins described above. Typically, the nucleic acid sequence is DNA. As described above, when unnatural amino acids are biosynthetically incorporated into proteins, one route can be to use codons such as TAA, TGA, or TGG, which normally code for protein chain termination (so-called “nonsense” codons) for incorporation of such amino acids. In that event, the nucleic acid sequence can include one or more of such “nonsense” codons under circumstances in which they do not result in chain termination. When such codons are intended to be used for the introduction of a translatable unnatural amino acid, they need to be conserved in any variant of the sequence.

DNA sequences encoding the mutated proteins or fusion proteins of the invention, including native, truncated, and extended polypeptides, can be obtained by several methods. For example, the DNA can be isolated using hybridization procedures that are well known in the art. These include, but are not limited to: (1) hybridization of probes to genomic or cDNA libraries to detect shared nucleotide sequences, (2) antibody screening of expression libraries to detect shared structural features; and (3) synthesis by the polymerase chain reaction (PCR). RNA sequences of the invention can be obtained by methods known in the art (See, for example, Current Protocols in Molecular Biology, Ausubel, et al., Eds., 1989).

The development of specific DNA sequences encoding mutated proteins or fusion proteins of the invention can be obtained by: (1) isolation of a double-stranded DNA sequence from the genomic DNA; (2) chemical manufacture of a DNA sequence to provide the necessary codons for the polypeptide of interest; and (3) in vitro synthesis of a double-stranded DNA sequence by reverse transcription of mRNA isolated from a eukaryotic donor cell. In the latter case, a double-stranded DNA complement of mRNA is eventually formed which is generally referred to as CDNA. Of these three methods for developing specific DNA sequences for use in recombinant procedures, the isolation of genomic DNA is the least common. This is especially true when it is desirable to obtain the microbial expression of mammalian polypeptides due to the presence of introns. The synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the formation of cDNA sequences. Among the standard procedures for isolating cDNA sequences of interest is the formation of plasmid-carrying cDNA libraries which are derived from reverse transcription of mRNA which is abundant in donor cells that have a high level of genetic expression. When used in combination with polymerase chain reaction technology, even rare expression products can be clones. In those cases where significant portions of the amino acid sequence of the polypeptide are known, the production of labeled single or double-stranded DNA or RNA probe sequences duplicating a sequence putatively present in the target CDNA may be employed in DNA/DNA hybridization procedures which are carried out on cloned copies of the cDNA which have been denatured into a single-stranded form (Jay, et al., Nucleic Acid Research 11:2325, 1983).

With respect to nucleotide sequences that are within the scope of the invention, all nucleotide sequences encoding the polypeptides that are embodiments of the invention as described are included in nucleotide sequences that are within the scope of the invention. This further includes all nucleotide sequences that encode polypeptides according to the invention that incorporate conservative amino acid substitutions as defined above. This is with the proviso that, when the nucleic acid sequence includes one or more “nonsense” codons under circumstances in which they do not result in chain termination and are intended to be used for the introduction of a translatable unnatural amino acid, these nonsense codons need to be conserved in any variant of the sequence.

Nucleic acid sequences of the present invention further include nucleic acid sequences that are at least 95% identical to the sequences above, with the proviso that the nucleic acid sequences retain the activity of the sequences before substitutions of bases are made, including any activity of proteins that are encoded by the nucleotide sequences and any activity of the nucleotide sequences that is expressed at the nucleic acid level, such as the binding sites for proteins affecting transcription. Preferably, the nucleic acid sequences are at least 97.5% identical. More preferably, they are at least 99% identical. For these purposes, “identity” is defined according to the Needleman-Wunsch algorithm (S. B. Needleman & C. D. Wunsch, “A General Method Applicable to the Search for Similarities in the Amino Acid Sequence of Two Proteins,” J. Mol. Biol. 48: 443-453 (1970)).

Nucleotide sequences encompassed by the present invention can also be incorporated into a vector, including, but not limited to, an expression vector, and used to transfect or transform suitable host cells, as is well known in the art. The vectors incorporating the nucleotide sequences that are encompassed by the present invention are also within the scope of the invention. Host cells that are transformed or transfected with the vector or with polynucleotides or nucleotide sequences of the present invention are also within the scope of the invention. The host cells can be prokaryotic or eukaryotic; if eukaryotic, the host cells can be mammalian cells, insect cells, or yeast cells. If prokaryotic, the host cells are typically bacterial cells.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl2 or RbCl can be used. Transformation can also be performed after forming a protoplast of the host cell or by electroporation.

When the host is a eukaryote such methods of transfection of DNA as calcium phosphate co-precipitates, conventional mechanical procedures such as microinjection, electroporation, insertion of a plasmid encased in liposomes, or virus vectors may be used.

A variety of host-expression vector systems may be utilized to express the mutated protein or fusion protein coding sequence. These include but are not limited to microorganisms such as bacteria transformed with recombinant bacteriophage DNA, plasmid DNA or cosmid DNA expression vectors containing a mutated protein or fusion protein coding sequence; yeast transformed with recombinant yeast expression vectors containing the mutated protein or fusion protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a mutated protein or fusion protein coding sequence; insect cell systems infected with recombinant virus expression vectors (e.g., baculovirus) containing a mutated protein or fusion protein coding sequence; or animal cell systems infected with recombinant virus expression vectors (eg., retroviruses, adenovirus, vaccinia virus) containing a mutated protein or fusion protein coding sequence, or transformed animal cell systems engineered for stable expression. In such cases where glycosylation may be important, expression systems that provide for translational and post-translational modifications may be used; e.g., mammalian, insect, yeast or plant expression systems

Depending on the host/vector system utilized, any of a number of suitable transcription and translation elements, including constitutive and inducible promoters, transcription enhancer elements, transcription terminators, etc. may be used in the expression vector (see e.g., Bitter, et al., Methods in Enzymology, 153:516-544, 1987). For example, when cloning in bacterial systems, inducible promoters such as pL of bacteriophage λ, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the inserted mutated protein or fusion protein coding sequence.

In bacterial systems a number of expression vectors may be advantageously selected depending upon the use intended for the mutated protein or fusion protein expressed. For example, when large quantities are to be produced, vectors which direct the expression of high levels of fusion protein products that are readily purified may be desirable. Those which are engineered to contain a cleavage site to aid in recovering the protein are preferred. Such vectors include but are not limited to the Escherichia coli expression vector pUR278 (Ruther, et al., EMBO J., 2:1791, 1983), in which the mutated protein or fusion protein coding sequence may be ligated into the vector in frame with the lac Z coding region so that a hybrid (mutated protein or fusion protein)-lac Z protein is produced; pIN vectors (Inouye & Inouye, Nucleic Acids Res. 13:3101-3109, 1985; Van Heeke & Schuster, J. Biol. Chem. 264:5503-5509, 1989); and the like.

In yeast, a number of vectors containing constitutive or inducible promoters may be used. For a review see, Current Protocols in Molecular Biology, Vol. 2, 1988, Ed. Ausubel, et al., Greene Publish. Assoc. & Wiley lnterscience, Ch. 13; Grant, et al., 1987, Expression and Secretion Vectors for Yeast, in Methods in Enzymology, Eds. Wu & Grossman, 31987, Acad. Press, N.Y., Vol. 153, pp.516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. DM Glover, 1986, IRL Press, Wash., D.C.). Alternatively, vectors may be used which promote integration of foreign DNA sequences into the yeast chromosome. Fungi, in general, can be used for expression of proteins using appropriate expression vectors.

In cases where plant expression vectors are used, the expression of a mutated protein or fusion protein coding sequence may be driven by any of a number of promoters. For example, viral promoters such as the 35S RNA and 19S RNA promoters of CaMV (Brisson₅ et al., Nature, 310:511-514, 1984), or the coat protein promoter to TMV (Takamatsu, et al., EMBO J., 6:307-311, 1987) may be used; alternatively, plant promoters such as the small subunit of RUBISCO (Coruzzi, et al., EMBO J. 3:1671-1680, 1984; Broglie, et al., Science 224:838-843, 1984); or heat shock promoters, e.g., soybean hspl7.5-E or hsp 17.3-B (Gurley, et al., Mol. Cell. Biol., 6:559-565, 1986) may be used. These constructs can be introduced into plant cells using Ti plasmids, Ri plasmids, plant virus vectors, direct DNA transformation, microinjection, electroporation, etc. For reviews of such techniques see, for example, Weissbach & Weissbach, Methods for Plant Molecular Biology, Academic Press, NY, Section VIII, pp. 421-463, 1988; and Grierson & Corey, Plant Molecular Biology, 2d Ed., Blackie, London, Ch. 7-9, 1988.

An alternative expression system that can be used to express a protein of the invention is an insect system. In one such system, Autographa californica nuclear polyhedrosis virus (AcNPV) is used as a vector to express foreign genes. The virus grows in Spodoptera frugiperda cells. The mutated protein or fusion protein polypeptide coding sequence may be cloned into non-essential regions (Spodoptera frugiperda for example the polyhedrin gene) of the virus and placed under control of an AcNPV promoter (for example the polyhedrin promoter). Successful insertion of the mutated protein or fusion protein coding sequence will result in inactivation of the polyhedrin gene and production of non-occluded recombinant virus (i.e., virus lacking the proteinaceous coat coded for by the polyhedrin gene). These recombinant viruses are then used to infect cells in which the inserted gene is expressed. (E.g., see Smith, et al., J. Biol. 46:584, 1983; Smith, U.S. Pat. No. 4,215,051).

Eukaryotic systems, and preferably mammalian expression systems, allow for proper post-translational modifications of expressed mammalian proteins to occur. Therefore, eukaryotic cells, such as mammalian cells that possess the cellular machinery for proper processing of the primary transcript, glycosylation, phosphorylation, and, advantageously secretion of the gene product, are the preferred host cells for the expression of a mutated protein or fusion protein, particularly when it is desired to substantially retain the original glycosylation pattern of Fc domains or portions thereof. Such host cell lines may include but are not limited to CHO, VERO, BHK, HeLa, COS, MDCK, 293, and WI38.

Mammalian cell systems that utilize recombinant viruses or viral elements to direct expression may be engineered. For example, when using adenovirus expression vectors, the coding sequence of a mutated protein or fusion protein may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the mutated protein or fusion protein in infected hosts (e.g., see Logan & Shenk, Proc. Nati. Acad. Sci. USA 81:3655-3659, 1984). Alternatively, the vaccinia virus 7.5K promoter may be used. (e.g., see, Mackett, et al., Proc. Natl. Acad. Sci. USA, 79:7415-7419, 1982; Mackett, et al., J. Virol. 49:857-864, 1984; Panicali, et al., Proc. Natl. Acad. Sci. USA, 79:4927-4931, 1982). Of particular interest are vectors based on bovine papilloma virus which have the ability to replicate as extrachromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of the mutated protein or fusion protein gene in host cells (Cone & Mulligan, Proc. Natl. Acad. Sci. USA 81:6349-6353, 1984). High level expression may also be achieved using inducible promoters, including, but not limited to, the metallothionine IIA promoter and heat shock promoters.

For long-term, high-yield production of recombinant proteins, stable expression is preferred. Rather than using expression vectors which contain viral origins of replication, host cells can be transformed with a cDNA controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including but not limited to the herpes simplex virus thymidine kinase (Wigler, et al., Cell 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes, which can be employed in tk.sup,-, hgprt.sup.- or aprt.sup.- cells respectively. Also, antimetabolite resistance-conferring genes can be used as the basis of selection; for example, the genes for dhfr, which confers resistance to methotrexate (Wigler, et al., NatI. Acad. Sci. USA,77:3567, 1980; O'Hare, et al., Proc. Nati. Acad. Sci. USA, 78:1527, 1981); gpt, which confers resistance to mycophenolic acid (Mulligan & Berg, Proc. Natl. Acad. Sci. USA, 78:2072, 1981; neo, which confers resistance to the aminoglycoside G418 (Colberre-Garapin, et al., J. Mol. Biol., 150:1, 1981); and hygro, which confers resistance to hygromycin (Santerre, et al., Gene, 30:147, 1984). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:804, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory ed., 1987).

Accordingly, another aspect of the invention is vectors incorporating nucleic acid segments encoding mutated proteins or fusion proteins according to the present invention.

Yet another aspect of the invention is host cells transformed or transfected with such vectors.

Still another aspect of the invention is a method for producing a mutated protein or a fusion protein according to the invention, the method comprising the steps of:
(1) culturing a transformed or transfected host cell as described above under conditions such that the mutated protein or fusion protein is expressed; and
(2) isolating the mutated protein or fusion protein from the transformed or transfected host cell to produce the protein.

Methods for the isolation of mutated proteins or fusion proteins are well known in the art and need not be described further in detail herein, For example, methods such as precipitation with salts such as ammonium sulfate, ion exchange chromatography, gel filtration, affinity chromatography, electrophoresis, isoelectric focusing, isotachophoresis, chromatofocusing, and other techniques are well known in the art and are described in R. K. Scopes, “Protein Purification: Principles and Practice” (3^{rd} ed., Springer-Verlag, New York, 1994).

The mutated protein or fusion protein that is produced can be used to generate a labeled protein according to the techniques described above.

### EXAMPLE

The invention is illustrated by the following Example. This Example is for illustrative purposes only and is not intended to limit the invention.

Libraries were prepared where three or four randomized amino acids were appended to the amino-terminus of an Fc region and selected the libraries for Fc's that covalently bound to the following reactive compounds: (B=biotin HPDP; M=maleimide biotin; I=iodoacetyl biotin; H=Halotag). Following selections, the clones were sequenced. All clones were from the initial panning with targets coated on the plates except for those labeled “rp” for repeat panning. Those were incubated with the compounds in solution lacking BSA then placed on a well coated with streptavidin and blocked with BSA. If an asterisk (*) and (tag) is shown, this means that a Q (glutamine) appears in the expressed protein.

The results are shown in Table 1.

TABLE 1

| Sample Name | Description | Selected Amino Acids |
|---|---|---|
| RPF2356 | pC3X FcRan3 vs. B#3 | CWE |
| RPF2357 | pC3X FcRan3 vs. B#7 | HQC |
| RPF2457 | pC3X FcRan4rp vs. (B)M #4 | HAC (P del) |
| RPF2458 | pC3X FcRan4rp vs. (B)M #6 | RSG |
| RPF2460 | pC3X FcRan4rp vs. (B)M #10 | VLA |
| RPF2358 | pC3X FcRan3 vs. M#9 | TVR |
| RPF2456 | pC3X FcRan4rp vs. B(M) #2 | II* (tag) |
| RPF2359 | pC3X FcRan3 vs. BM#10 | MHN |
| RPF2459 | pC3X FcRan4rp vs. B(M) #8 | *VLM (tag) |
| RPF2468 | pC3X FcRan3rp vs. B(M) #7 | GLVG |
| RPF2362 | pC3X FcRan4 vs. B#5 | f/s YTCS/LYVF |
| RPF2363 | pC3X FcRan3 vs. I#2 | AHT |
| RPF2364 | pC3X FcRan4 vs. I#6 | AGR (P del) |
| RPF2461 | pC3X FcRan4rp vs. I#2 | HWL |
| RPF2462 | pC3X FcRan4rp vs. I#4 | f/s IGC/LAV |
| RPF2463 | pC3X FcRan4rp vs. I#8 | TM* (tag) |
| RPF2464 | pC3X FcRan4rp vs. I#10 | APH |
| RPF2365 | pC3X FcRan4 vs. I#9 | SVW*(tag) |
| RPF2469 | pC3X FcRan3rp vs. I#3 | *FSV (tag) |
| RPF2366 | pC3X FcRan3 vs. H#6 | WPP |
| RPF2465 | pC3X FcRan4rp vs. H#1 | DA* (tag) |
| RPF2466 | pC3X FcRan4rp vs. H#3 | *LV (tag) |
| RPF2467 | pC3X FcRan4rp vs. H#10 | CLC (pt mut) |
| RPF2367 | pC3X FcRan3 vs. H#8 | WLSF |
| RPF2368 | pC3X FcRan3 vs. H#9 | YRVL |
| RPF2369 | pC3X FcRan4 vs. H#10 | CF*W (tag) |
| RPF2470 | pC3X FcRan3rp vs. H#10 | QLPH |

The clones listed in bold in Table 1 were chosen based on their sequence and independently expressed and shown to bind compounds using ELISA. A wide range of sequences can be selected using this approach by varying the number of randomized residues and the nature of the reactive compound.

### Advantages of the Invention

The present invention provides a powerful and versatile method for the Fc portion of antibody molecules and related molecules including Fc regions for immunostaining and immunotargeting. The methods provide labeled molecules with less perturbation of conformation or activity of the labeled proteins than currently-available methods. The methods are flexible and have broad application, allowing labeling with a variety of linkers or without a linker, and allow the incorporation of labeled molecules into larger fusion proteins. Methods according to the present invention can exploit a modular approach to labeling so that both the amino- and carboxyl-termini of labeled proteins can be bound to desirable proteins or domains.

Methods according to the present invention allow selection and production of mutated proteins for labeling using phage display methods.

The present invention also provides for the use of labeled proteins in diagnosis and treatment. Labeled proteins according to the present invention can be used either in vitro or in vivo in a large number of diagnostic procedures, including immunostaining and immunolabeling. Labeled cells can be sorted, detected, and quantitated using fluorescence-activated cell sorting (FACS) or other techniques. Labeled proteins according to the present invention can also be used in methods of treatment and can be formulated into pharmaceutical compositions.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a”, “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

I claim:

1. A method for labeling a protein molecule that includes therein the Fc portion of an antibody molecule comprising the steps of:
(a) providing a protein molecule that includes therein the Fc portion of an antibody molecule, the molecule having an amino-terminal serine residue;
(b) oxidizing the amino-terminal serine residue to an aldehyde group; and
(c) reacting the protein molecule with a targeting molecule including therein a moiety reactive with an aldehyde to produce a labeled protein molecule such that the targeting molecule solely directs the targeting of the labeled protein molecule to a target that is a soluble molecule or a cell-surface molecule.

2. A method for labeling a protein molecule that includes therein the Fc portion of an antibody molecule comprising the steps of:
(a) providing a protein molecule that includes therein the Fc portion of an antibody molecule, the molecule having at least one amino acid including therein a side chain with aldehyde or keto functionality; and
(b) reacting the aldehyde or keto functionality of the protein molecule with a targeting molecule including therein a group reactive with an aldehyde or keto functionality to produce a labeled protein molecule such that the targeting molecule solely directs the targeting of the labeled protein molecule to a target that is a soluble molecule or a cell-surface molecule.

3. A method for labeling a protein molecule that includes therein the Fc portion of an antibody molecule comprising the steps of:
(a) providing a protein molecule that includes therein the Fc portion of an antibody molecule, the protein molecule having a reactive amino acid residue selected from the group consisting of an azide-substituted amino acid residue and an alkyne-substituted amino acid residue;
(b) providing a targeting molecule, the targeting molecule having a reactive residue selected from the group consisting of an azide and an alkyne- such that the protein molecule and the targeting molecule, taken together, have an azide and an alkyne; and
(c) reacting the protein molecule with the targeting molecule by azide-alkyne [3+2] cycloaddition to produce a labeled protein molecule such that the targeting molecule solely directs the targeting of the labeled protein molecule to a target that is a soluble molecule or a cell-surface molecule.

4. A method for labeling a protein molecule that includes therein the Fc portion of an antibody molecule comprising the steps of:
(a) providing a protein molecule that includes therein the Fc portion of an antibody molecule, the molecule having at least one amino acid including therein a side chain with azido functionality; and
(b) in a Staudinger ligation reaction, reacting the azido functionality of the protein molecule with a targeting molecule that is covalently linked to an ortho-disubstituted aromatic moiety, one substituent being carbomethoxy and the other substitutent being diphenylphosphino, to produce a labeled protein molecule, such that the labeled protein molecule has one substituent of the aromatic moiety being diphenylphosphinyl and the other substituent being a carboxamide moiety, with the nitrogen of the carboxamide moiety being linked to the protein molecule such that the targeting molecule solely directs the targeting of the labeled protein molecule to a target that is a soluble molecule or a cell-surface molecule.

5. A method for labeling a protein molecule that includes therein the Fc portion of an antibody molecule comprising the steps of:
(a) providing a protein molecule that includes therein the Fc portion of an antibody molecule, the molecule having an amino acid selected from the group consisting of p-acetylphenylalanine and m-acetylphenylalanine; and
(b) reacting the amino acid selected from the group consisting of p-acetylphenylalanine and m-acetylphenylalanine of the protein molecule with a targeting molecule containing a reactive moiety selected from the group consisting of a hydrazide, an alkoxyamine, and a semicarbazide to produce a labeled protein molecule such that the targeting molecule solely directs the targeting of the labeled protein molecule to a target that is a soluble molecule or a cell-surface molecule.

6. A method for labeling a protein molecule that includes therein the Fc portion of an antibody molecule comprising the steps of:
(a) providing a protein molecule that includes therein the Fc portion of an antibody molecule, the protein molecule having a reactive amino acid residue reactive with an electrophile;
(b) providing a targeting molecule that includes an electrophile reactive with the amino acid residue; and
(c) reacting the targeting molecule with the protein molecule by reacting the reactive amino acid residue with the electrophile to produce the labeled protein molecule such that the targeting molecule solely directs the targeting of the labeled protein molecule to a target that is a soluble molecule or a cell-surface molecule.

7. A method for labeling a protein molecule that includes therein the Fc portion of an antibody molecule comprising the steps of:
(a) providing a protein molecule that includes therein the Fc portion of an antibody molecule, the protein molecule having a reactive amino acid residue including therein an electrophilic group reactive with a nucleophile;
(b) providing a targeting molecule that includes a nucleophile reactive with the amino acid residue; and
(c) reacting the targeting molecule with the protein molecule by reacting the reactive amino acid residue with the nucleophile to produce the labeled protein molecule such that the targeting molecule solely directs the targeting of the labeled protein molecule to a target that is a soluble molecule or a cell-surface molecule.

8. A method for labeling a protein molecule that includes therein the Fc portion of an antibody molecule comprising the steps of;
(a) providing a protein molecule that includes therein the Fc portion of an antibody molecule, the protein molecule having a mutated haloalkane dehalogenase domain therein, the mutated haloalkane dehalogenase domain having therein an aspartate residue, the side chain of the aspartate residue being capable of esterification; and
(b) reacting the protein molecule with a targeting molecule having a reactive haloalkane moiety to form a stable ester to produce a labeled protein molecule such that the targeting molecule solely directs the targeting of the labeled protein molecule to a target that is a soluble molecule or a cell-surface molecule.

9. A method for labeling a protein molecule that includes therein the Fc portion of an antibody molecule comprising the steps of; (a) providing a protein molecule that includes therein the Fc portion of an antibody molecule, the protein molecule having a reactive aldehyde residue;
(b) reacting the aldehyde residue with a bifunctional hydroxylamine linker having two H₂N—O— moieties, the aldehyde residue forming a C═N bond with one of the moieties; and
(c) reacting the other H₂N—O— moiety of the bifunctional hydroxylamine linker with a targeting molecule having a diketone moiety to produce a labeled protein molecule such that the targeting molecule solely directs the targeting of the labeled protein molecule to a target that is a soluble molecule or a cell-surface molecule.

10. A method for labeling a protein molecule that includes therein the Fc portion of an antibody molecule comprising the steps of:
(a) providing a protein molecule that includes therein the Fc portion of an antibody molecule, the protein molecule having a first reactive amino acid at its amino-terminus and a second reactive amino acid at its carboxyl-terminus;
(b) reacting a first molecule selected from the group consisting of a targeting molecule and a component of a fusion protein with the first reactive amino acid to link the first molecule to the protein molecule; and
(c) reacting a second molecule selected from the group consisting of a targeting molecule and a component of a fusion protein with the second reactive amino acid to link the second molecule to the protein molecule;
with the proviso that the first reactive amino acid does not react with the second reactive amino acid and such that the targeting molecule solely directs the targeting of the labeled protein molecule to a target that is a soluble molecule or a cell-surface molecule, with the proviso that at least one targeting molecule is coupled.

11. The method of claim 1 wherein the amino-terminal serine is oxidized to an aldehyde function by oxidation with periodate to a glyoxylyl residue.

12. The method of claim 1 wherein the protein molecule is the Fc domain of an antibody molecule.

13. The method of claim 2 wherein the protein molecule is the Fc domain of an antibody molecule.

14. The method of claim 3 wherein the protein molecule is the Fc domain of an antibody molecule.

15. The method of claim 4 wherein the protein molecule is the Fc domain of an antibody molecule.

16. The method of claim 5 wherein the protein molecule is the Fc domain of an antibody molecule.

17. The method of claim 6 wherein the protein molecule is the Fc domain of an antibody molecule.

18. The method of claim 7 wherein the protein molecule is the Fc domain of an antibody molecule.

19. The method of claim 8 wherein the protein molecule is the Fc domain of an antibody molecule.

20. The method of claim 9 wherein the protein molecule is the Fc domain of an antibody molecule.

21. The method of claim 10 wherein the protein molecule is the Fc domain of an antibody molecule.

22. The method of claim 1 wherein the protein molecule is produced by site-directed mutagenesis of a naturally-occurring protein molecule such that the amino-terminal residue is mutated to a reactive serine or cysteine.

23. The method of claim 1 wherein the targeting molecule comprises: (i) a targeting module; (ii) a linker covalently linked to the targeting module; and (iii) a reactive module covalently linked to the linker, the reactive module including therein a hydroxylamine moiety or derivative thereof.

24. The method of claim 2 wherein the targeting molecule comprises: (i) a targeting module; (ii) a linker covalently linked to the targeting module; and (iii) a reactive module covalently linked to the linker, the reactive module including therein a hydroxylamine moiety or derivative thereof.

25. The method of claim 3 wherein the targeting molecule comprises: (i) a targeting module; (ii) a linker covalently linked to the targeting module; and (iii) a reactive module covalently linked to the linker, the reactive module reacting with the protein.

26. The method of claim 4 wherein the targeting molecule comprises: (i) a targeting module; (ii) a linker covalently linked to the targeting module; and (iii) a reactive module covalently linked to the linker, the reactive module reacting with the protein.

27. The method of claim 5 wherein the targeting molecule comprises: (i) a targeting module; (ii) a linker covalently linked to the targeting module; and (iii) a reactive module covalently linked to the linker, the reactive module reacting with the protein.

28. The method of claim 6 wherein the targeting molecule comprises: (i) a targeting module; (ii) a linker covalently linked to the targeting module; and (iii) a reactive module covalently linked to the linker, the reactive module reacting with the protein.

29. The method of claim 7 wherein the targeting molecule comprises: (i) a targeting module; (ii) a linker covalently linked to the targeting module; and (iii) a reactive module covalently linked to the linker, the reactive module reacting with the protein.

30. The method of claim 8 wherein the targeting molecule comprises: (i) a targeting module; (ii) a linker covalently linked to the targeting module; and (iii) a reactive module covalently linked to the linker, the reactive module reacting with the protein.

31. The method of claim 1 wherein the targeting molecule specifically targets an integrin.

32. The method of claim 2 wherein the targeting molecule specifically targets an integrin.

33. The method of claim 3 wherein the targeting molecule specifically targets an integrin.

34. The method of claim 4 wherein the targeting molecule specifically targets an integrin.

35. The method of claim 5 wherein the targeting molecule specifically targets an integrin.

36. The method of claim 6 wherein the targeting molecule specifically targets an integrin.

37. The method of claim 7 wherein the targeting molecule specifically targets an integrin.

38. The method of claim 8 wherein the targeting molecule specifically targets an integrin.

39. The method of claim 9 wherein the targeting molecule specifically targets an integrin.

40. The method of claim 10 wherein the targeting molecule specifically targets an integrin.

41. A labeled protein molecule produced by the method of claim 1.

42. A labeled protein molecule produced by the method of claim 2.

43. A labeled protein molecule produced by the method of claim 3.

44. A labeled protein molecule produced by the method of claim 4.

45. A labeled protein molecule produced by the method of claim 5.

46. A labeled protein molecule produced by the method of claim 6.

47. A labeled protein molecule produced by the method of claim 7.

48. A labeled protein molecule produced by the method of claim 8.

49. A labeled protein molecule produced by the method of claim 9.

50. A labeled protein molecule produced by the method of claim 10.

51. A mutated protein incorporating an altered amino acid at the amino-terminus of the sequence of the protein, the protein including therein the Fc portion of an antibody molecule, the mutated protein being reactive with a targeting molecule that has a group reactive with the altered amino acid at the amino-terminus such that the targeting molecule directs the targeting of the mutated protein covalently linked to the targeting molecule to a target.

52. The mutated protein of claim 51 wherein the altered amino acid after mutation is selected from the group consisting of serine, cysteine, lysine, histidine, methionine, aspartate, and glutamate.

53. The mutated protein of claim 52 wherein the altered amino acid after mutation is serine and the targeting molecule includes a hydroxylamine, hydrazine, hydrazide, or derivative thereof.

54. The mutated protein of claim 52 that is a fusion protein.

55. A mutated protein including the Fc portion of an antibody molecule and incorporating therein a non-naturally-occurring amino acid, the non-naturally-occurring amino acid being selected from the group consisting of:
(a) an azide-substituted amino acid;
(b) an alkyne-substituted amino acid;
(c) p-acetylphenylalanine;
(d) m-acetylphenylalanine;
(e) β-oxo-α-aminobutyric acid; and
(f) (2-ketobutyl)-tyrosine;
wherein the non-naturally-occurring amino acid is located such that the mutated protein can be covalently linked to a targeting molecule such that the targeting molecule solely directs the targeting of the mutated protein covalently linked to the targeting molecule to a target that is a soluble molecule or a cell-surface molecule.

56. The mutated protein of claim 55 that is a fusion protein.

57. A nucleic acid sequence encoding the protein of claim 51.

58. A nucleic acid sequence encoding the protein of claim 55.

59. A vector including the nucleic acid sequence of claim 57.

60. A vector including the nucleic acid sequence of claim 58.

61. A host cell transformed or transfected with the vector of claim 59.

62. A host cell transformed or transfected with the vector of claim 60.

63. A method for producing a mutated protein or fusion protein comprising the steps of:
(a) culturing the transformed or transfected host cell of claim 61 under conditions such that the mutated protein or fusion protein is expressed; and
(b) isolating the mutated protein or fusion protein from the transformed or transfected host cell to produce the protein.

64. A method for producing a mutated protein or fusion protein comprising the steps of:
(a) culturing the transformed or transfected host cell of claim 62 under conditions such that the mutated protein or fusion protein is expressed; and
(b) isolating the mutated protein or fusion protein from the transformed or transfected host cell to produce the protein.

65. A method of delivering a labeled protein molecule that effects a biological activity to cells, tissue, an extracellular matrix biomolecule or a biomolecule in the fluid of an individual, wherein the method comprises administering to the individual the labeled protein molecule of claim 41, wherein the labeled protein molecule is specific for the cells, tissue extracellular matrix biomolecule or fluid biomolecule and wherein the labeled protein molecule effects a biological activity.

66. A method of delivering a labeled protein molecule that effects a biological activity to cells, tissue, an extracellular matrix biomolecule or a biomolecule in the fluid of an individual, wherein the method comprises administering to the individual the labeled protein molecule of claim 42, wherein the labeled protein molecule is specific for the cells, tissue extracellular matrix biomolecule or fluid biomolecule and wherein the labeled protein molecule effects a biological activity.

67. A method of delivering a labeled protein molecule that effects a biological activity to cells, tissue, an extracellular matrix biomolecule or a biomolecule in the fluid of an individual, wherein the method comprises administering to the individual the labeled protein molecule of claim 43, wherein the labeled protein molecule is specific for the cells, tissue extracellular matrix biomolecule or fluid biomolecule and wherein the labeled protein molecule effects a biological activity.

68. A method of delivering a labeled protein molecule that effects a biological activity to cells, tissue, an extracellular matrix biomolecule or a biomolecule in the fluid of an individual, wherein the method comprises administering to the individual the labeled protein molecule of claim 44, wherein the labeled protein molecule is specific for the cells, tissue extracellular matrix biomolecule or fluid biomolecule and wherein the labeled protein molecule effects a biological activity.

69. A method of delivering a labeled protein molecule that effects a biological activity to cells, tissue, an extracellular matrix biomolecule or a biomolecule in the fluid of an individual, wherein the method comprises administering to the individual the labeled protein molecule of claim 45, wherein the labeled protein molecule is specific for the cells, tissue extracellular matrix biomolecule or fluid biomolecule and wherein the labeled protein molecule effects a biological activity.

70. A method of delivering a labeled protein molecule that effects a biological activity to cells, tissue, an extracellular matrix biomolecule or a biomolecule in the fluid of an individual, wherein the method comprises administering to the individual the labeled protein molecule of claim 46, wherein the labeled protein molecule is specific for the cells, tissue extracellular matrix biomolecule or fluid biomolecule and wherein the labeled protein molecule effects a biological activity.

71. A method of delivering a labeled protein molecule that effects a biological activity to cells, tissue, an extracellular matrix biomolecule or a biomolecule in the fluid of an individual, wherein the method comprises administering to the individual the labeled protein molecule of claim 47, wherein the labeled protein molecule is specific for the cells, tissue extracellular matrix biomolecule or fluid biomolecule and wherein the labeled protein molecule effects a biological activity.

72. A method of delivering a labeled protein molecule that effects a biological activity to cells, tissue, an extracellular matrix biomolecule or a biomolecule in the fluid of an individual, wherein the method comprises administering to the individual the labeled protein molecule of claim 48, wherein the labeled protein molecule is specific for the cells, tissue extracellular matrix biomolecule or fluid biomolecule and wherein the labeled protein molecule effects a biological activity.

73. A method of delivering a labeled protein molecule that effects a biological activity to cells, tissue, an extracellular matrix biomolecule or a biomolecule in the fluid of an individual, wherein the method comprises administering to the individual the labeled protein molecule of claim 49, wherein the labeled protein molecule is specific for the cells, tissue extracellular matrix biomolecule or fluid biomolecule and wherein the labeled protein molecule effects a biological activity.

74. A method of delivering a labeled protein molecule that effects a biological activity to cells, tissue, an extracellular matrix biomolecule or a biomolecule in the fluid of an individual, wherein the method comprises administering to the individual the labeled protein molecule of claim 50, wherein the labeled protein molecule is specific for the cells, tissue extracellular matrix biomolecule or fluid biomolecule and wherein the labeled protein molecule effects a biological activity.

75. A pharmaceutical composition comprising:
(a) the labeled protein of claim 41 in an effective amount; and
(b) a pharmaceutically acceptable carrier.

76. A pharmaceutical composition comprising:
(a) the labeled protein of claim 42 in an effective amount; and
(b) a pharmaceutically acceptable carrier.

77. A pharmaceutical composition comprising:
(a) the labeled protein of claim 43 in an effective amount; and
(b) a pharmaceutically acceptable carrier.

78. A pharmaceutical composition comprising:
(a) the labeled protein of claim 44 in an effective amount; and
(b) a pharmaceutically acceptable carrier.

79. A pharmaceutical composition comprising:
(a) the labeled protein of claim 45 in an effective amount; and
(b) a pharmaceutically acceptable carrier.

80. A pharmaceutical composition comprising:
(a) the labeled protein of claim 46 in an effective amount; and (b) a pharmaceutically acceptable carrier.

81. A pharmaceutical composition comprising:
(a) the labeled protein of claim 47 in an effective amount; and
(b) a pharmaceutically acceptable carrier.

82. A pharmaceutical composition comprising:
(a) the labeled protein of claim 48 in an effective amount; and
(b) a pharmaceutically acceptable carrier.

83. A pharmaceutical composition comprising:
(a) the labeled protein of claim 49 in an effective amount; and
(b) a pharmaceutically acceptable carrier.

84. A pharmaceutical composition comprising:
(a) the labeled protein of claim 50 in an effective amount; and
(b) a pharmaceutically acceptable carrier.
